(19) United States
(12) Patent Application Publication
(10) Pub. No.: US 2020/0197463 A1
(43) Pub. Date: Jun. 25, 2020

(54) NOVEL CANNABIS LINES AND EXTRACTS WITH ANTI-INFLAMMATORY POTENCIES

(71) Applicants: Pathway RX Inc., Lethbridge (CA); Inplanta Biotechnology Inc., Lethbridge (CA)

(72) Inventors: Olga Kovalchuk, Lethbridge (CA); Dongping Li, Lethbridge (CA); Anna Kovalchuk, Lethbridge (CA); Dwight Darryl Hudson, Raymond (CA); Igor Kovalchuk, Lethbridge (CA)

(21) Appl. No.: 16/713,030

(22) Filed: Dec. 13, 2019

Related U.S. Application Data
(60) Provisional application No. 62/781,656, filed on Dec. 19, 2018.

(51) Int. Cl. A61K 36/185

(52) U.S. Cl. CPC A61K 36/185

(57) ABSTRACT

The present invention provides new unique cannabis lines, extracts and methods for their use in anti-inflammatory therapies and modalities. The method includes generation of unique lines, whole plant extract preparation, treating normal human 3D tissues with UV to induce inflammation, and then with extracts in amount sufficient to profoundly down-regulate inflammation and molecular pathways involved in rheumatoid arthritis (RA), irritable bowel disease (IBD) and other auto-inflammatory disorders. The modulation of these pathways is a key to treatment success in RA, IBD and the other auto-inflammatory disorders.

### FIELD OF THE INVENTION

The present invention relates generally to products and methods for treating inflammation, and more specifically to methods and products for treating inflammation from cannabis and hemp plants.

### BACKGROUND OF THE INVENTION

In 2017, the USA Centers for Disease Control and Prevention reported that 54.4 million adults (25% of US population) suffer from arthritis—inflammation of joints in the body. Arthritis is characterized by pains, swelling and stiffness of the joints, and is a leading cause of disability. The main types of arthritis are rheumatoid arthritis, osteoarthritis, gout, lupus arthritis, psoriatic arthritis, as well as fibromyalgia.

Of those, rheumatoid arthritis (RA) is one of the most severe ones. It results in significant mortality and morbidity and has a major socio-economic impact (1). RA is an autoimmune disease characterized by joint pain, swelling, stiffness and progressive loss of joint function as well as damage throughout the body. It is a very serious long-term disease with limited treatment options, and rather poor outcomes (1). RA is a clinical syndrome that encompasses several disease subsets that entail several inflammatory cascades, but all eventually result in a final common pathway whereby constant synovial inflammation leading to damage of joint cartilage and underlying bone.

Similarly, irritable bowel disease (IBD), represented by Crohn's disease and ulcerative colitis, is yet another chronic disease with large inflammatory component. Many components of the mucosal immune system are involved in the pathogenesis of IBD and include intestinal epithelial cells, innate lymphoid cells, cells of the innate (macrophages/monocytes, neutrophils, and dendritic cells) and adaptive (T-cells and B-cells) immune system, and their secreted mediators (cytokines and chemokines) (Wallace et al., 2014).

The main underlying molecular feature of RA and IBD is a molecular pathway driving overexpression and overproduction of tumor necrosis factor (TNF), which drives synovial inflammation and joint damage.

TNF alpha is an inflammatory cytokine that stimulates and maintains cellular activation and migration of leukocytes to inflammatory sites. TNF acts though binding to its receptors (TNFR) that are located throughout the body. Interaction of TNF with receptors causes increased expression of other cytokines (IL-1 and IL-6) and chemokines, which, in turn, activate leukocytes, suppresses regulatory T cells, causes production of MMP proteins which degrade tissues, induces apoptosis, and also has some anti-tumor effects (2).

Along with RA where TNF pathway is a main molecular driver, there are several other auto-inflammatory diseases that constitute a rare group conditions characterized by recurrent fevers, systemic inflammation, and dysfunctions of the innate immune system. These are Familial Mediterranean Fever, cryopyrin-associated periodic syndrome, mevalonate kinase, deficiency/hyperimmunoglobulinemia D Syndrome, TNF receptor-associated auto-inflammatory syndrome, and systemic juvenile idiopathic arthritis/adult-onset Still's disease. All of the aforementioned conditions are characterized by overproduction or deficiency of inhibition of various cytokines (2).

Since TNF has been recognized as the main mediator of inflammation, and regulation of immune inflammation results in significant alleviation of RA symptoms, TNF has become a key target for anti-RA and anti-auto-inflammatory disease treatments and modalities. Different drugs to block TNF activity have been developed, and five TNF inhibitors have been approved by the FDA to treat a variety of inflammatory conditions. Albeit, these drugs have limited efficacy, cause very significant side effects, and bare a huge price tag (2,3). Along with TNF, interleukins (IL-1, IL-6, IL-8, IL-18 and others) as well as other cytokines are important treatment targets. Several IL inhibitors are currently in clinical use, but those also have significant side effects and rather limited efficacy (2).

Therefore, new approaches are needed that can efficiently and swiftly block TNF and inflammatory cytokine cascades and thus curb inflammation and lead to disease remission. There thus remains an unmet need to provide effective non-toxic methods and products for treating inflammation in mammalian subjects.

### SUMMARY OF THE INVENTION

It is an object of some aspects of the present invention to provide methods and products for blocking TNF and inflammatory cytokine cascades and thus curbing inflammation, thereby leading to inflammatory disease remission

In some embodiments of the present invention, improved methods and products are provided for blocking TNF and inflammatory cytokine cascades and thus curb inflammation, thereby leading to inflammatory disease remission.

In other embodiments of the present invention, a method and product is described for treating inflammatory diseases and disorders in mammalian subjects.

In other embodiments of the present invention, a method and product is described for downregulating TNF and inflammatory cytokine cascades thereby treating inflammatory diseases and disorders in human subjects.

It is an object of some aspects of the present invention to provide compositions for improving wellness in a human or mammalian organism.

It is another object of some aspects of the present invention to provide compositions for preventing or treating diseases or disorders in a human or mammalian organism.

The compositions and dosage forms of the present invention are useful in promoting health and preventing or treating a large number of disorders in human patients and other mammalian subjects.

In additional embodiments of the present invention, compositions and methods are provided for treating and/or preventing inflammatory disorders.

The present invention is directed to compositions and methods for treating disorders, in general, and more particularly, inflammatory diseases/disorders. The compositions of the present invention may be used for improving wellness of a human or mammalian subject. Additionally, the compositions of the present invention may be used to treat any disorder or ailment in a human patient or mammalian subject. Furthermore, the compositions of the present invention may be conveniently used in conjunction with a drug to treat any disorder or ailment in a human patient or mammalian subject.

In other embodiments of the present invention, a method and product is described for protecting a mammalian body from inflammatory disorders and diseases.

In other embodiments of the present invention, a method and product is described for healing a mammalian body from inflammatory disorders and diseases.

Some embodiments of the present invention provide compounds, compositions and formulations from at least one of hemp and cannabis.

Some further embodiments of the present invention provide methods for downregulating at least one inflammation pathway gene.

Some further embodiments of the present invention provide methods for downregulating at least one inflammation pathway gene product.

Some further embodiments of the present invention provide methods for downregulating expression of at least one RA-related gene.

Some further embodiments of the present invention provide methods for downregulating at least one RA-related gene product.

Some further embodiments of the present invention provide methods for downregulating expression of at least one IBD-related gene.

Some further embodiments of the present invention provide methods for downregulating at least one IBD-related gene product.

Some further embodiments of the present invention provide methods for downregulating expression of at least one inflammatory pathway gene.

Some further embodiments of the present invention provide methods for downregulating expression of at least one gene from TNF gene family.

Some further embodiments of the present invention provide methods for downregulating expression of at least one of IL, CCL and CXCL gene.

Some further embodiments of the present invention provide methods for downregulating expression of STATS gene product.

Some further embodiments of the present invention provide methods for downregulating expression of COX-2 gene product.

Some further embodiments of the present invention provide methods for upregulating expression of SOCS-3 gene product.

Some further embodiments of the present invention provide methods for downregulating expression of BCL-2 gene product.

Some further embodiments of the present invention provide methods for downregulating at least one interleukin gene product.

Some further embodiments of the present invention provide methods for downregulating at least one inflammatory pathway gene product.

Some further embodiments of the present invention provide methods for downregulating at least TNF gene product.

The present invention provides new unique cannabis lines, extracts and methods for their use in anti-inflammatory therapies and modalities. The method includes generation of unique lines, whole plant extract preparation, treating normal human 3D tissues with UV or with TNFα or TNFα+IFNγ or treating normal human intestinal cells with TNFα+IFNγ to induce inflammation, and then with extracts in amount sufficient to profoundly down-regulate inflammation and molecular pathways involved in RA, IBD and other auto-inflammatory disorders. The modulation of these pathways is a key to treatment success in RA, IBD and other auto-inflammatory disorders.

The present invention provides new Cannabis sativa lines and extracts and a method of using them as a means to down-regulate inflammation and molecular cascades, which drive RA, IBD and other auto-inflammatory diseases of muscular-skeletal system. The disclosure also provides methods of modulating inflammation through the application of extracts of novel cannabis lines to tissue models.

Accordingly, the present invention provides a method for modulating inflammatory gene expression by cannabis extracts (e.g., in skin tissues after exposure to UV light or intestinal tissues after exposure to TNFα+IFNγ, known inflammation-inducing agents) by providing amounts sufficient to modulate gene expression where modulation of gene expression results in suppression of RA and IBD pathways and inflammation, and consequently, of a disease state.

Here, several freshly prepared extracts of Cannabis sativa lines (designated lines #4, #6, #8, #10, #12, #13, #14, #15, #18, #24, #28, #30 and #81) were used. Most extracts displayed anti-TNF, anti-inflammatory activity (lines #4, #6, #8, #10, #13, #14, #18, #24, #28, #30 and #81).

Using EpiDermFT human 3D skin tissue models exposed to UV to induce inflammation and then treated with extracts of new cannabis lines, it was shown that several new extracts strongly down-regulated expression of rheumatoid arthritis pathway genes—TNF, IL genes, CCL and CXCL pro-inflammatory genes, which modulate inflammation, immunity and autoimmunity, and apoptosis.

Using Epilntestinal human 3D intestinal tissue models exposed to TNFα+INFγ to induce inflammation and then treated with extracts of new cannabis lines, it was shown that several new extracts strongly down-regulated expression of RA and IBD pathway genes—TNF, IL genes, COX-2, STAT-3, BCL-2, p65, CCL, CXCL pro-inflammatory genes, which modulate inflammation, immunity and autoimmunity, and apoptosis and upregulating the expression of SOCS-3.

The present invention provides a potent anti-inflammatory, anti-TNF and anti-IL activity of novel cannabis line extracts, and presents a novel and promising natural resource for anti-RA and anti-IBD treatments, and for treatments of other types of arthritis and other aforementioned auto-inflammatory disorders.

### NON-LIMITING EMBODIMENTS OF THE PRESENT INVENTION

1. A method for treating inflammation, the method comprising:
a) combining at least one marijuana or hemp cultivar and at least one other marijuana or hemp cultivar to form at least one Cannabis line;
b) extracting at least one compound from said at least one Cannabis line to form an extract; and
c) treating at least one of a mammalian subject and an in vitro model with at least one of said extract and said at least one compound in an effective amount to treat said inflammation.

2. A method according to embodiment 1, wherein said treating step comprises providing an effective amount of said extract or said at least one compound to said mammalian subject or to said in vitro model to modulate gene expression.

3. A method according to embodiment 1, wherein said modulation of gene expression comprises modulating at least one gene selected from the group consisting of: a TNF pathway gene, a TNFR gene, an Interleukin gene, a chemokine gene, a gene associated with an inflammatory disease, a gene associated with an inflammatory disorder, a gene associated with a leukocyte, a gene associated with a body joint, a gene associated with synovial fluid, a gene associated with intestine, and combinations thereof.

4. A method according to embodiment 3, wherein said modulation of gene expression results in at least one of a reduction or an increase of 0.1-3 log₂ fold change in expression of said at least one gene.

5. A method according to embodiment 4, wherein said at least one Cannabis line is selected from the group consisting of a marijuana/marijuana hybrid line, hemp/hemp hybrid line and hemp/marijuana hybrid line.

6. A method according to embodiment 5, wherein said at least one line is selected from the group consisting of designated lines #4, #6, #8, #10, #13, #14, #18, #24, #28, #30 and #81.

7. A method according to embodiment 1, wherein said extracting step comprises extracting flowers of said at least one Cannabis line.

8. A method according to embodiment 7, wherein said extracting step comprises extracting said at least one compound in at least one organic solvent.

9. A method according to embodiment 6, wherein said extracting step is performed at a temperature in the range of 15-to 60° C. and at a pressure in a range of −0.5 to 1.5 bar and wherein said at least one organic solvent comprises ethyl acetate.

10. A method according to embodiment 4, wherein said at least one gene is selected from the group consisting of: TNF, TNFAIP3, TNFAIP6, TNFRSF1B, TNIP1, TNIP3, ILIA, IL1B, IL1R2, IL1RN, IL23A, IL24, IL32, IL36G, IL411, IL6, IL7R, CCL2, CCL20, CXCL2, CXCL3, CXCL5, CXCL6 and CXCL8, COX-2, SOCS-3, STAT-3, BCL-2 and combinations thereof.

11. A method according to embodiment 10, wherein said at least one Cannabis line comprises line #4.

12. A method according to embodiment 4, wherein said at least one gene is selected from the group consisting of: TNF, TNFAIP3, TNFRSF1B, IL1A, IL1B, IL1R2, IL20, IL23A, IL32 and IL6 and combinations thereof.

13. A method according to embodiment 12, wherein said at least one Cannabis line comprises line #6.

14. A method according to embodiment 4, wherein said at least one gene is selected from the group consisting of: TNF, TNFAIP3, TNFAIP6, TNFRSF10D, TNFRSF12A, TNFRSF1B, TNIP1, TRAF1, IL11, IL13RA2, ILIA, IL1B, IL1RN, IL20, IL23A, IL24, IL32, IL36G, IL411, IL6, CXCL1, CXCL2, CXCL3, CXCL5, CXCL6, CXCL8, COX-2, SOCS-3, STAT-3, BCL-2 and combinations thereof.

15. A method according to embodiment 14, wherein said at least one Cannabis line comprises line #8.

16. A method according to embodiment 4, wherein said at least one gene is selected from the group consisting of: TNF, TNFAIP3, TNFAIP6, TNFRSF10D, TNFRSF12A, TNFRSF1B, TNIP1, TRAF1, IL11, IL13RA2, ILIA, IL1B, IL1RN, IL20, IL23A, IL24, IL32, IL36G, IL411, IL6, CXCL1, CXCL2, CXCL3, CXCL5, CXCL6, CXCL8, COX-2, SOCS-3, STAT-3, BCL-2 and combinations thereof.

17. A method according to embodiment 16, wherein said at least one Cannabis line comprises line #10.

18. A method according to embodiment 4, wherein said at least one gene is selected from the group consisting of: TNFRSF10, TUF0, IL20RA, IL20RB, IL22RA1, IL33, IL36RN, IL37 and CXCL16 and combinations thereof.

19. A method according to embodiment 18, wherein said at least one Cannabis line comprises line #12.

20. A method according to embodiment 4, wherein said at least one gene is selected from the group consisting of: IL11, IL13RA2, ILIA, ILR2, IL20, IL23A, IL33, IL36G, IL36RN, IL7R, TNF, TNFAIP3, TNFRSF10D, TNFRSF12A, TNIP1, TNIP3, CCL2, CCL20, CXCL2, CXCL5 and CXCL6 and combinations thereof.

21. A method according to embodiment 20, wherein said at least one Cannabis line comprises line #13.

22. A method according to embodiment 4, wherein said at least one gene is selected from the group consisting of: TNF, TNFAIP3, TNFAIP6, TNFRSF12A, TNFRSF1B, ILIA, ILR2, IL23A, IL32, IL36G, IL6, CCL2, CCL20, CXCL2, COX-2, SOCS-3, STAT-3, BCL-2 and combinations thereof.

23. A method according to embodiment 22, wherein said at least one Cannabis line comprises line #14.

24. A method according to embodiment 4, wherein said at least one gene is selected from the group consisting of: TNF, TNFAIP3, TNFAIP6, TNFRSF12A, TNFRSF1B, ILIA, ILR2, IL23A, IL32, IL36G, IL6, CCL2, CCL20, CXCL2, COX-2, SOCS-3, STAT-3, BCL-2 and combinations thereof.

25. A method according to embodiment 24, wherein said at least one Cannabis line comprises line #18.

26. A method according to embodiment 4, wherein said at least one gene is selected from the group consisting of: TNF, TNFAIP3, TNFAIP6, TNFRSF12A, TNFRSF1B, ILIA, ILR2, IL23A, IL32, IL36G, IL6, CCL2, CCL20, CXCL2, COX-2, SOCS-3, STAT-3, BCL-2 and combinations thereof.

27. A method according to embodiment 26, wherein said at least one Cannabis line comprises line #24.

28. A method according to embodiment 4, wherein said at least one gene is selected from the group consisting of: TNF, TNFAIP3, TNFAIP6, TNFRSF12A, TNFRSF1B, ILIA, ILR2, IL23A, IL32, IL36G, IL6, CCL2, CCL20, CXCL2, COX-2, SOCS-3, STAT-3, BCL-2 and combinations thereof.

29. A method according to embodiment 28, wherein said at least one Cannabis line comprises line #28.

30. A method according to embodiment 4, wherein said at least one gene is selected from the group consisting of: TNF, TNFAIP3, TNFAIP6, TNFRSF12A, TNFRSF1B, ILIA, ILR2, IL23A, IL32, IL36G, IL6, CCL2, CCL20, CXCL2, COX-2, SOCS-3, STAT-3, BCL-2 and combinations thereof.

31. A method according to embodiment 30, wherein said at least one Cannabis line comprises line #30.

32. A method according to embodiment 4, wherein said at least one gene is selected from the group consisting of: TNF, TNFAIP3, TNFAIP6, TNFRSF12A, TNFRSF1B, ILIA, ILR2, IL23A, IL32, IL36G, IL6, CCL2, CCL20, CXCL2, COX-2, SOCS-3, STAT-3, BCL-2 and combinations thereof.

33. A method according to embodiment 32, wherein said at least one Cannabis line comprises line #81.

34. A method according to embodiment 4, wherein said at least one gene is selected from the group consisting of: CXCL5 and TIMP4 and combinations thereof.

35. A method according to embodiment 34, wherein said at least one Cannabis line comprises line #15.

36. A method according to embodiment 1, wherein said at least one compound is provided in a concentration in a range of 0.0001-0.05 μg/μl, 0.001-0.05 μg/μl, 0.001-0.005 μg/μl, 0.003-0.03 μg/μl or 0.007-0.015 μg/μl.

37. A method according to embodiment 1, wherein said at least one compound is provided in a solvent extract and said solvent extract exhibits inflammation healing properties.

38. A method according to embodiment 37, wherein said solvent extract is at least 2-20, 3-15, 4-12, 5-10 or 6-9 times as effective as at least one of THC and CBD, administered at the same concentration in treating said disease.

39. A method according to embodiment 1, wherein said Cannabis line is a Cannabis sativa line.

40. An organic extract of at least one plant line, said at least one plant line formed from combining at least one of:
a) at least one marijuana or hemp cultivar; and
b) at least one other marijuana or hemp cultivar,

wherein said organic extract comprises at least one compound suitable for treating an inflammatory mammalian disease or disorder.

41. An organic extract according to embodiment 40, wherein said at least one plant line comprises a Cannabis sativa line.

42. An organic extract according to embodiment 41, wherein said mammalian inflammatory disease or disorder is selected from the group consisting of arthritis, rheumatoid arthritis, psoriatic arthritis, osteoarthritis, Familial Mediterranean Fever, cryopyrin-associated periodic syndrome, is mevalonate kinase, deficiency/hyperimmunoglobulinemia D Syndrome, a TNF receptor-associated autoinflammatory syndrome, systemic juvenile idiopathic arthritis/adult-onset Still's disease, fibromyalgia, Crohn's disease, ulcerative colitis, inflammation, an allergy and combinations thereof.

43. An organic extract according to embodiment 40, wherein said organic extract is at least 2-20, 3-15, 4-12, 5-10 or 6-9 times as effective as at least one of THC and CBD, administered at the same concentration in treating said disease.

44. A combination therapy, isolated from an organic extract of at least one hybrid line, said at least one hybrid line formed from combining at least one of:
a) at least one marijuana cultivar; and
b) at least one hemp cultivar; and

wherein said organic extract comprises a plurality of compounds suitable for treating a mammalian inflammatory disease or disorder.

45. A combination therapy according to embodiment 44, wherein said mammalian inflammatory disease or disorder m is selected from the group consisting of: arthritis, rheumatoid arthritis, psoriatic arthritis, osteoarthritis, Familial Mediterranean Fever, cryopyrin-associated periodic syndrome, is mevalonate kinase, deficiency/hyperimmunoglobulinemia D Syndrome, a TNF receptor-associated autoinflammatory syndrome, systemic juvenile idiopathic arthritis/adult-onset Still's disease, fibromyalgia, Crohn's disease, ulcerative colitis, inflammation, an allergy and combinations thereof

46. A line of Cannabis sativa formed by combining at least one marijuana or hemp cultivar and at least one other marijuana or hemp cultivar, said line to be deposited at a publicly available culture collection, designated herein #4, #6, #8, #10, #13, #14, #18, #24, #28, #30 and #81.

The present invention will be more fully understood from the following detailed description of the preferred embodiments thereof, taken together with the drawings.

### BRIEF DESCRIPTION OF THE DRAWINGS

The invention will now be described in connection with certain preferred embodiments with reference to the following illustrative figures so that it may be more fully understood.

With specific reference now to the figures in detail, it is stressed that the particulars shown are by way of example and for purposes of illustrative discussion of the preferred embodiments of the present invention only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the context of this application, the word “Figure” is abbreviated to “Fig”.

FIGS. 1A-1M are high performance liquid chromatography (HPLC) profiles of tested lines, in accordance with some embodiments of the present invention;

FIG. 2 shows a simplified schematic, part-pictorial illustration of a method for identifying new anti-inflammatory lines and extracts, in accordance with some embodiments of the present invention;

FIG. 3 shows a simplified schematic, part-pictorial illustration of a method using EpiDermFt tissues to detect anti-inflammatory properties of extracts. A. EpiDermFT has normal skin tissue structure with differentiated dermis and epidermis and is constructed from human-derived epidermal keratinocytes and dermal fibroblasts. It exhibits in vivo-like growth and morphological characteristics whereby cells sustain differentiation and metabolic status similar to those of human epidermis. B. Tissue insert in a well with medium. C. Scheme of UV-induced inflammation experiment, in accordance with some embodiments of the present invention;

FIG. 4 shows the effects of line #4 on the expression of TNF, IL and other pro-inflammatory molecules. Genes with a False Discovery Rate (FDR)-adjusted p-value<0.05 and log2 fold change>0.6 (1.5× change) were considered differentially expressed. Changes in the levels of gene expression are shown as log2 fold as compared to control, in accordance with some embodiments of the present invention;

FIG. 5 shows the effects of line #6 on the expression of TNF, IL and other pro-inflammatory molecules. Genes with a False Discovery Rate (FDR)-adjusted p-value<0.05 and log2 fold change>0.6 (1.5× change) were considered differentially expressed. Changes in the levels of gene expression are shown as log2 fold as compared to control, in accordance with some embodiments of the present invention;

FIG. 6 shows the effects of line #8 on the expression of TNF, IL and other pro-inflammatory molecules. Genes with a False Discovery Rate (FDR)-adjusted p-value<0.05 and log2 fold change>0.6 (1.5× change) were considered differentially expressed. Changes in the levels of gene expression are shown as log2 fold as compared to control, in accordance with some embodiments of the present invention;

FIG. 7 shows the effects of line #12 on the expression of TNF, IL and other pro-inflammatory molecules. Genes with a False Discovery Rate (FDR)-adjusted p-value<0.05 and log2 fold change>0.6 (1.5× change) were considered differentially expressed. Changes in the levels of gene expression are shown as log2 fold as compared to control, in accordance with some embodiments of the present invention;

FIG. 8 shows the effects of line #13 on the expression of TNF, IL and other pro-inflammatory molecules. Genes with a False Discovery Rate (FDR)-adjusted p-value<0.05 and log2 fold change>0.6 (1.5× change) were considered differentially expressed. Changes in the levels of gene expression are shown as log2 fold as compared to control, in accordance with some embodiments of the present invention;

FIG. 9 shows effects of line #14 on the expression of TNF, IL and other pro-inflammatory molecules. Genes with a False Discovery Rate (FDR)-adjusted p-value<0.05 and log2 fold change>0.6 (1.5× change) were considered differentially expressed. Changes in the levels of gene expression are shown as log2 fold as compared to control, in accordance with some embodiments of the present invention;

FIG. 10 shows the effects of line #15 on the expression of TNF, IL and other pro-inflammatory molecules. Genes with a False Discovery Rate (FDR)-adjusted p-value<0.05 and log2 fold change>0.6 (1.5× change) were considered differentially expressed. Changes in the levels of gene expression are shown as log2 fold as compared to control, in accordance with some embodiments of the present invention;

FIG. 11 shows a simplified pictorial illustration of a method using EpiIntestinal tissues to detect anti-inflammatory properties of extracts. A. Epilntestinal has normal small intestine epithelial cell structure and is constructed from human-derived columnar epithelial and endothelial cells. It exhibits in vivo-like growth and morphological characteristics whereby cells sustain differentiation and metabolic status similar to those of human intestinal epithelium. B. Tissue insert in a well with medium. C. Scheme of TNF/INF-induced inflammation experiment, in accordance with some embodiments of the present invention;

FIGS. 12A-12J show an induction of inflammation response by TNF/IFN and effects of various extracts on TNF, IL and other pro-inflammatory molecules. Genes with a False Discovery Rate (FDR)-adjusted p-value<0.05 were considered differentially expressed. Changes in the levels of gene expression are shown as log2 fold as compared to control. TNF/IFN, in accordance with some embodiments of the present invention; FIG. 12A. Induction of expression of TNF, interleukin and chemokine-related genes in response to TNF/IFN; FIG. 12B. Combined gene expression analysis of genes involved in inflammation (see Table 1 for the full list). “TNF/IFN”—gene expression in response to TNF/IFN (24 h after treatment). “TNF/IFN-DMSO”—persistence of the expression of pro-inflammatory genes 24 h after removal of TNF/IFN and media change (DMSO); FIGS. 12C-21J. Gene expression in response to various extracts. Extracts were given to cells exposed to TNF/IFN for 24 h. Expression analysis was performed 24 h after exposure to extracts, in accordance with some embodiments of the present invention;

FIG. 13A is a Western blot analysis of COX-2 and SOCS-3 after the exposure to TNF/INF and treatment with CBD or crude flower extracts (used as a control), in accordance with some embodiments of the present invention;

HSIEC, human small intestinal epithelial cells were used for the experiments. Cells were grown in normal media (Vehicle-) for 12 h or treated for 6 h with TNFα (40 ng/ml)/IFNγ (5 ng/ml) (Vehicle+) followed by 6 h of 10 μM CBD or different flower extracts. Cells were then harvested and protein expression was tested, in accordance with some embodiments of the present invention;

FIG. 13B shows a graph of quantification of the data for COX-2 using Image-J. GAPDH was used as a control. Bars shows standard error, calculated from 3 independent blots. Asterisks show significant difference from TNF/IFN induction, in accordance with some embodiments of the present invention;

FIG. 13C shows a graph of quantification of the data for SOCS-3 using Image-J. GAPDH was used as a control. Bars shows standard error, calculated from 3 independent blots. Asterisks show significant difference from TNF/IFN induction, in accordance with some embodiments of the present invention; and

FIG. 14 is a schematic part-pictorial representation of a method for developing novel treatments for RA, autoimmune and inflammatory diseases, in accordance with some embodiments of the present invention;

In all the figures similar reference numerals identify similar parts.

### DETAILED DESCRIPTION OF THE EMBODIMENTS

In the detailed description, numerous specific details are set forth in order to provide a thorough understanding of the invention. However, it will be understood by those skilled in the art that these are specific embodiments and that the present invention may be practiced also in different ways that embody the characterizing features of the invention as described and claimed herein.

The present invention provides new unique cannabis lines, extracts and methods for their use in anti-inflammatory therapies and modalities. The method includes generation of unique lines, whole plant extract preparation, treating normal human 3D tissues with UV to induce inflammation, and then with extracts in amount sufficient to profoundly down-regulate inflammation and molecular pathways involved in rheumatoid arthritis (RA), irritable bowel disease (IBD) and other auto-inflammatory disorders. The modulation of these pathways is a key to treatment success in RA and other auto-inflammatory disorders.

The present invention further provides methods of drug discovery. According to some embodiments, the method includes:
a) combining at least one marijuana cultivar and at least one hemp cultivar to form at least one hybrid line;
b) extracting at least one compound from said at least one hybrid line to form an extract; and
c) testing the extract in vitro to identify a biologically active extract.

The method further includes repeating steps a) to c) on a plurality of extracts to identify the most biologically active extracts. The method further includes isolating active compounds or components from the biologically active extracts. The method further comprises treating a patient with a disease or disorder with at least one of the active compounds, components or extracts to cure, alleviate or manage the disease or disorder.

### Materials and Methods

Plant Crude Extract Preparation:

Solvent used: Ethyl acetate ACS grade from Fisher cat# E145-4 (99.9% pure)

Extract Preparation: 3 g of the powdered plant tissue were weighed using an analytical balance Plant material was placed inside a 250 mL Erlenmeyer flask (clean). 100 mL of Ethyl Acetate was poured into the flask containing the plant material. The flasks were then wrapped with tin foil and shaken continuously (120 rpm) in an incubator @21° C. overnight and in the dark.

After overnight solvent extraction the extracts were filtered through cotton into a 100 mL round bottom flask. The extracts were concentrated to around 2-3 mL using a rotary vacuum evaporator. The extracts were then transferred to a tared 3 dram vial (cat# 60975L Kimble obtained from Fisher Scientific). The left over solvent was evaporated to dryness in an oven overnight @50° C. to eliminate the solvent completely. Mass of each extract was recoded.

Bioassay Preparation:

Preparation of 60 mg/mL Stocks.

The stocks were prepared weighing a 3-6 mg of crude extract into a micro centrifuge tube. The crude extract was dissolved in DMSO (Dimethyl sulfoxide anhydrous from Life technologies cat # D12345) to reach 60 mg/mL final concentration and stored at −20° C.

Preparation of Crude Extracts for Bioassay.

Appropriate cell culture media (in our experiments RPMI+10% FBS or EMEM+10% FBS) was used to dilute the 60 mg/mL stock. The stocks are allowed to thaw then added to the cell culture media, mixed thoroughly to ensure they are in solution and filtered through a 0.22 um syringe filter. These filtrates were ready to be applied to cells and tested.

Experimental Models:

EpiDerm full thickness 400 (EFT-400) Skin model (Mat Tek) was used as inflammation model. EpiDermFT has normal skin tissue structure with differentiated dermis and epidermis, and consists of normal, human epidermal keratinocytes (NHEK) and normal, human dermal fibroblasts (NHFB) cultured to form a multilayered model of the human dermis and epidermis. It exhibits in vivo-like growth and morphological characteristics whereby cells sustain differentiation and metabolic status similar to those of human epidermis. Tissues were equilibrated in EFT-400 for 24 h (overnight) then culture media EFT-400 was replaced and incubated for another 24 h.

Exposure and inflammation induction in EpiDerm by UVC:

Tissues were exposed for 2 min to UVC, in a biosafety cabinet. Distance from the light source was set to 10 cm; only 3 wells of the 6 well plate were exposed at a time (to make the distance effective in all 3 wells).

All crude extracts were diluted from a 60 mg/mL stock (the stock is prepared in DMSO). For this experiment a final concentration of 0.01 ug/uL in 30% glycerol-PBS was used. 24 h after UV exposure, 15 uL of 0.01 ug/uL extract solution or control (PBS alone) were applied to the tissue after exposure. Control samples consisted of the following samples: PBS-exposed only, PBS and DMSO added after UVC exposure (crude extracts are stored in DMSO).

Application volume of extracts: 15 uL of these solutions were applied on top of the tissues (inside the cup holding the tissue) ensuring even coverage of the tissue surface.

Tissues once treated were allowed to equilibrate at 37° C. in an incubator with 6% CO₂, for 48 hours. Then all tissues were frozen using liquid N₂ and stored at −80° C. and used to prepare RNA for sequencing.

Epilntestinal Skin—intestinal epithelial cell model (Mat Tek) was used as inflammation model. Epilntestinal is a 3D reconstructed tissue model produced from primary, human cell-derived small intestine epithelial and endothelial cells and fibroblasts. The highly differentiated tissue model is produced at the air-liquid-interface (ALI) in easy-to-handle tissue culture inserts. Structural analysis of the tissue model demonstrates columnar shaped basal cells and Kerckring folds. Ultrastructurally, Epilntestinal exhibits brush borders, functional tight junctions and mucous secreting granules, similar to in vivo tissue. It exhibits in vivo-like growth and morphological characteristics whereby cells sustain differentiation and metabolic status similar to those of human intestinal epithelium.

### Exposure and Inflammation Induction in Epilntestinal by TNF/IFN:

Tissues were equilibrated for 24 h (overnight) then culture media was replaced and incubated for another 24 h. Tissues were then exposed for 24 h to TNFα (40 ng/ml) and IFNγ (5 ng/ml) or to DMSO only, resulting in TNF/IFN″ or “Ct” samples, respectively.

All crude extracts were diluted from a 60 mg/mL stock (the stock is prepared in DMSO). After TNF/IFN exposure, 15 uL of crude extract solution or control (media with DMSO) were applied to the tissue after exposure, resulting in samples “#4”, “#6” etc. or “TNF/IFN+DMSO” samples, respectively. The samples then were collected for the analysis in 24 h after exposure.

Control samples were not treated and were used for the analysis of response to TNF/IFN (24 h). Group “TNF/IFN+DMSO” was used as a control for all extract treatments.

Application volume of extracts: 15 uL of these solutions were applied on top of the tissues (inside the cup holding the tissue) to the final concentration of the extract of 0.015 mg/ml ensuring even coverage of the tissue surface.

Tissues once treated were allowed to equilibrate at 37° C. in an incubator with 6% CO₂, for 24 hours. Then all tissues were frozen using liquid N₂ and stored at −80° C.

Gene Expression Profiling:

Three tissues per group were used for the analysis of gene expression profiles. RNA was extracted from tissues using TRIzol® Reagent (Invitrogen, Carlsbad, Calif.), further purified using an RNAesy kit (Qiagen), and quantified using Nanodrop2000c (ThermoScientific). Afterwards, RNA integrity and concentration were established using 2100 BioAnalyzer (Agilent). Sequencing libraries were prepared using Illumina's TruSeq RNA library preparation kits, and global gene expression profiles were determined using the Next 500 Illumina deep-sequencing platform at the University of Lethbridge Facility. Statistical comparisons between the control and treatment groups (all samples in triplicates) were performed using the DESeq Bioconductor package (version 1.8.3) and the baySeq Bioconductor package (version 1.10.0). Clustering of the samples was assessed with multidimensional scaling (MDS) plots built using the plotMDS function from the edgeR Bioconductor package. Features with a false discovery rate (FDR)<0.1 (10% false positive rate) were considered differentially expressed between conditions.

The functional annotations of differentially expressed genes were performed using David, GO (Gene Ontology) Elite, and GO-TermFinder. Pathways were visualized using Pathview/KEGG and DAVID bioinformatics platforms DAVID Bioinformatics Resources 6.7 KEGG Pathway platforms.

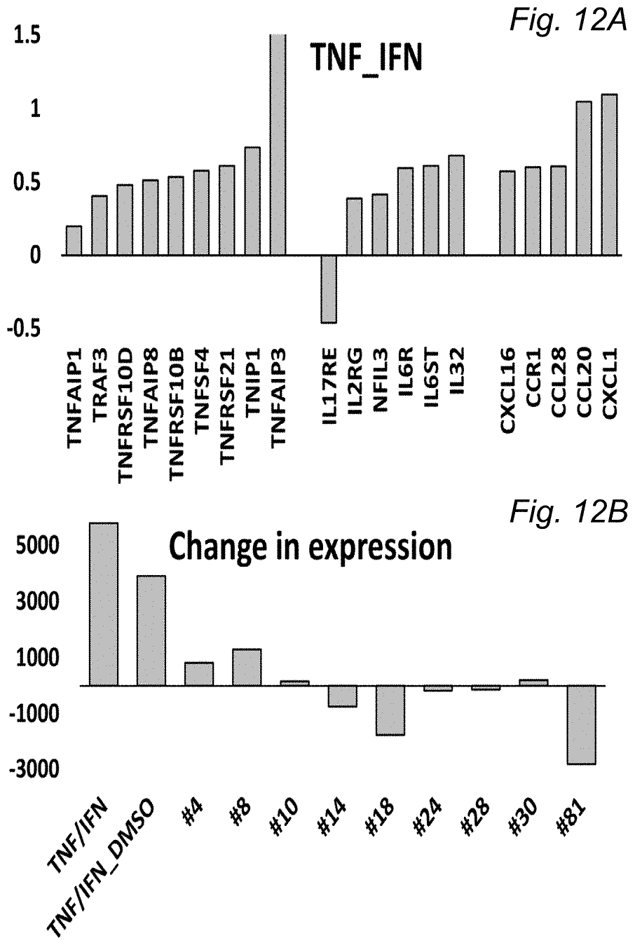

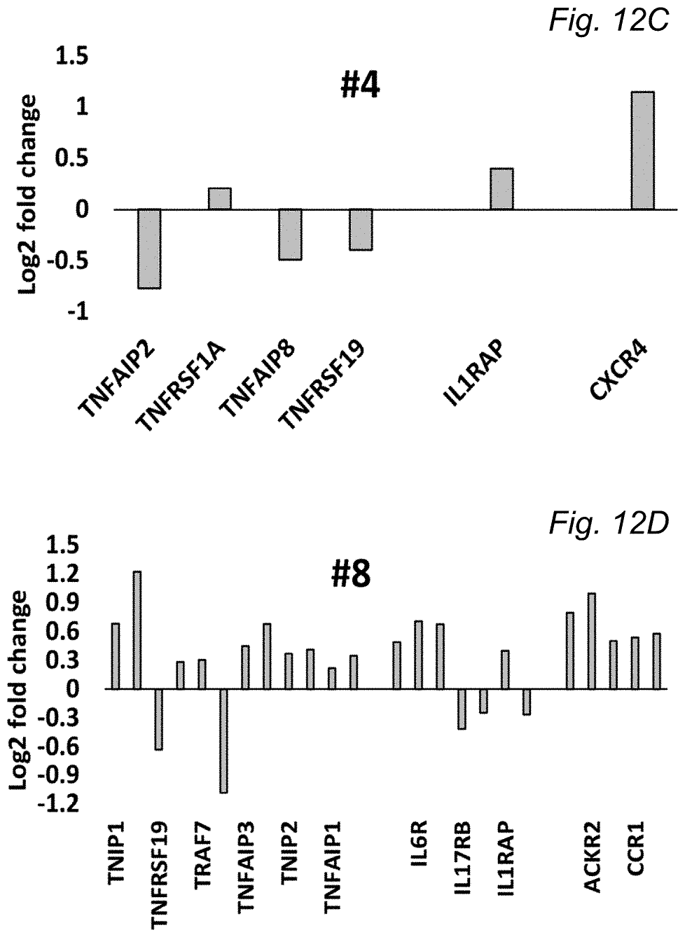

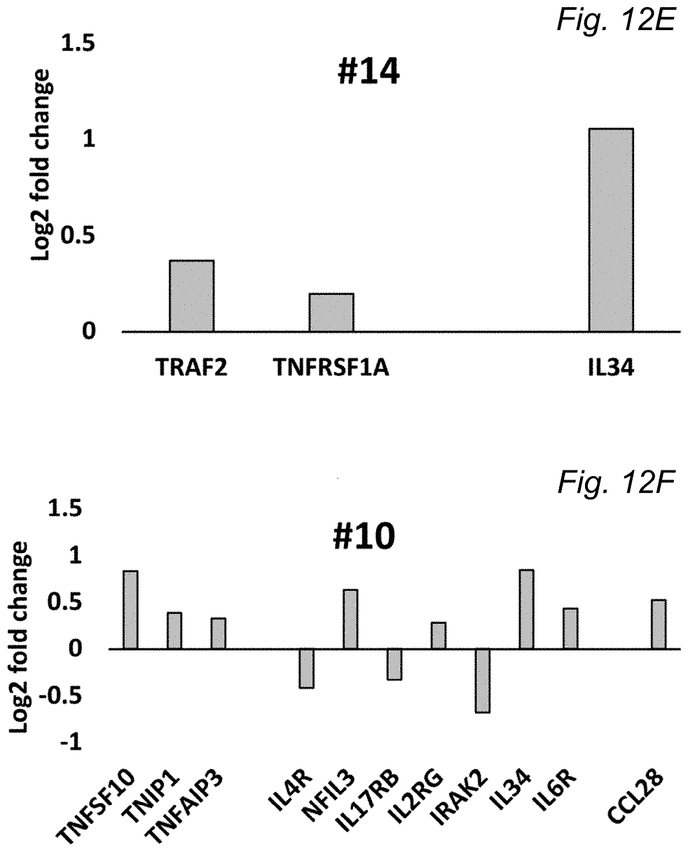

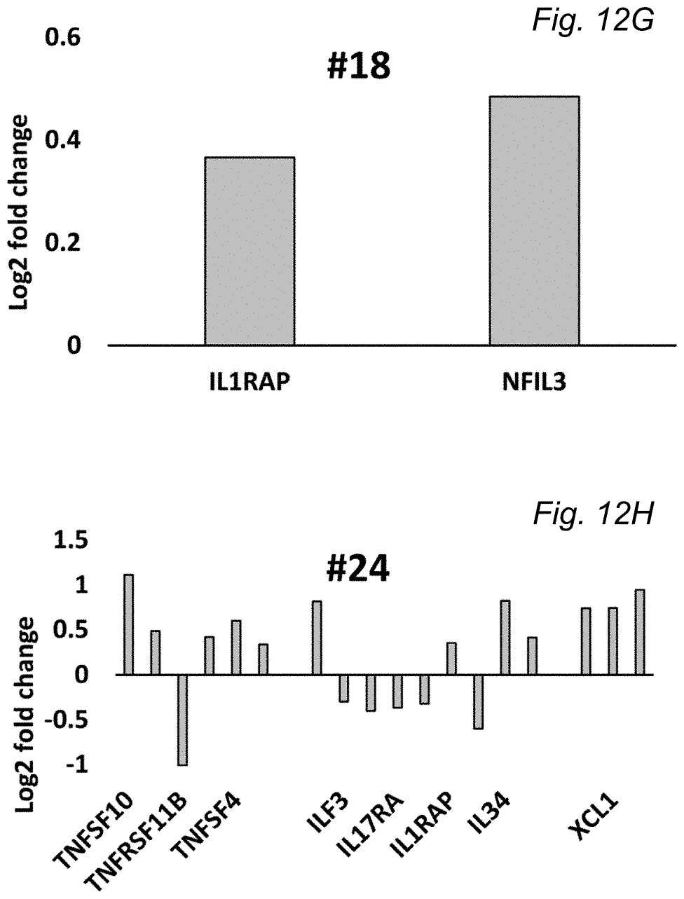

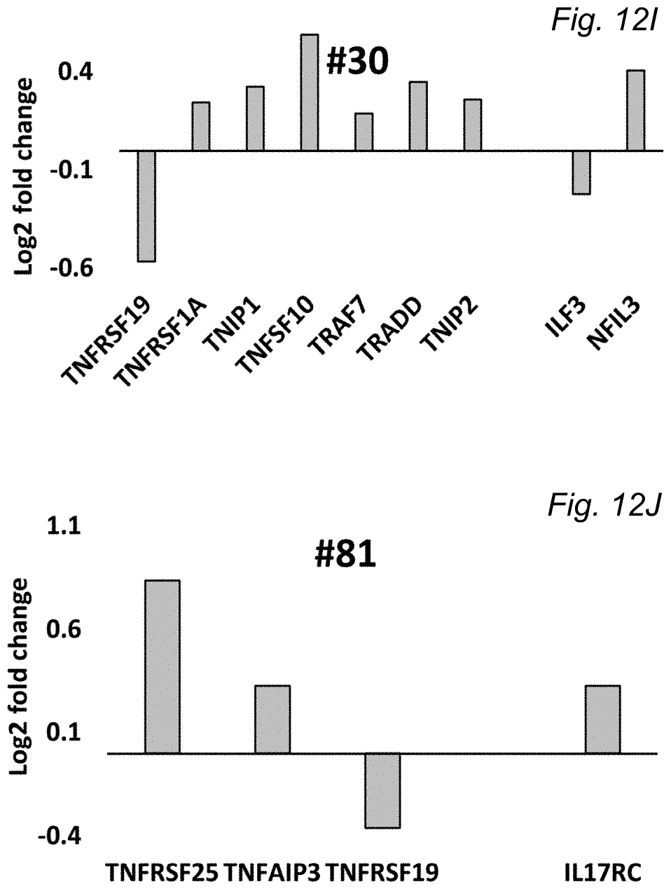

Next, the set of genes that are annotated were obtained as participants in ‘inflammatory response’ by Gene Ontology (Table 1). Then, the effect of the pairwise comparisons were quantified by adding the expression effect of the inflammatory genes from Table 1. A positive value indicates that the inflammatory pathway is overexpressed, while a negative value indicates that the inflammatory response pathway is repressed (FIG. 12B).

Proteomics Analysis:

The harvesting of tissues from Epilntestinal Small Intestine tissue Model (SMI-100) (MatTek) was done by removal of the membrane containing the tissue using a sterile scalpel blade. The tissue was separated from the membrane carefully with forceps. Then the tissue disc was split into 2 portions (one for protein and one for RNA work) and immediately frozen in liquid nitrogen and stored at −80C until processed. The cellular protein extracts were prepared in 100-150 ul RIPA lysis buffer (the cellular protein extracts were prepared in 60 ul RIPA lysis buffer, when cells harvested from 6-well plate) with 10 mM Tris-HCl (pH 7.5), 100 mM NaCl,1 mM EDTA, 1% Triton X-100, 10% glycerol, 0.1% SDS, 0.5% deoxycholate, 1 mM sodium orthovanadate, and 1 mM PMSF, and sonicated the cell. The protein solution was centrifugated at 12,000×g for 10 min.

The supernatant was transferred to a new microtube for store. Small aliquots (2 ul) of extracts were diluted with sterile ultra-pure water (ddH2O) in a 1:40 ratio and reserved for protein determination using protein assay reagents from Bio-Rad. 25 ul diluted protein solution was added in 1.25 Bio-Rad assay reagents followed by incubation at room temperature at 10 min. The mixture was analyzed immediately using the NanoDrop 2000/2000c Spectrophotometers (ThermoFisher Scientific Company, Wilmington, Del.). Equal amount of protein (60-100ug) were normalized with 4× loading buffer (0.0625M Tris, 2% SDS, 10% glycerol, 0.01% bromophenol blue and 1% 2-mercaptoethanol) and RIPA lysis buffer and heated at 95° C. for 10 mins. The protein sample was loaded in each well with a marker, PageRuler Plus Prestained Protein Ladder (Cat#26620, Thermo Scienctific, Massachusetts, USA), 10 to 250 kDa and separated by a 10% SDS-PAGE in slab gels of 10% polyacrylamide at 100V, and transferred to polyvinylidene difluoride membrane (GE Healthcare Biosciences). After the transfer, the membrane was blocked with 5% w/v non-fat dry milk for an hour.

Following the protocol, the membrane was incubated with the primary antibodies overnight. SOCS-3 protein was detected using a rabbit monoclonal SOCS-3 antibody (Cat#cs-2923, Cell Signaling Technologies, Massachusetts, United States) at 1:500. COX-2 protein was detected using a rabbit monoclonal COX-2 antibody (Cat#ab-62331, Abcam inc, Cambridge, United Kingdom) at 1:1000. STAT-3 protein was detected using a rabbit monoclonal STAT-3 antibody (Cat#sc-483, Santa Cruz biotechnology, Inc., Texas, United States) at 1:200. BCL-2 protein was detected using a rabbit monoclonal BCL-2 antibody (Cat#sc-7382, Santa Cruz biotechnology, Inc., Texas, United States) at 1:200.

As a loading control, GAPDH protein was detected using a mouse monoclonal GAPDH antibody (Cat#sc-47724, Santa Cruz biotechnology, Inc., Texas, United States) at 1:1000. After overnight incubation, the membrane was washed three times with 0.1% Tween-20 in PBS (PBS-T). The blot detected primary antibody binding with Bovine anti-Mouse secondary antibody (Cat#sc-2371, Santa Cruz biotechnology, Inc., Texas, United States) at 1:10000 or Donkey anti-Rabbit secondary antibodies (Cat#sc-2313, Santa Cruz biotechnology, Inc., Texas, United States) at 1:10000 following secondary washes with PBS-T. The membrane was exposed to ECL Prime Western Blotting System (Cat#GERPN2232, GE Healthcare, Chicago, USA) and the result is detected using the FluorChem HD2 Imaging System (Cell Biosciences, California, United States).

### Result Figures

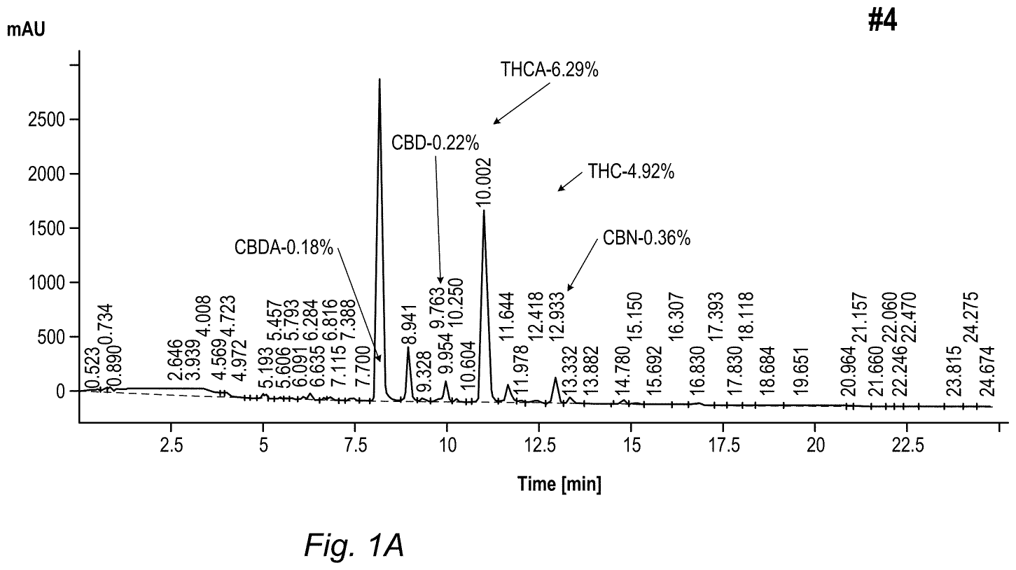

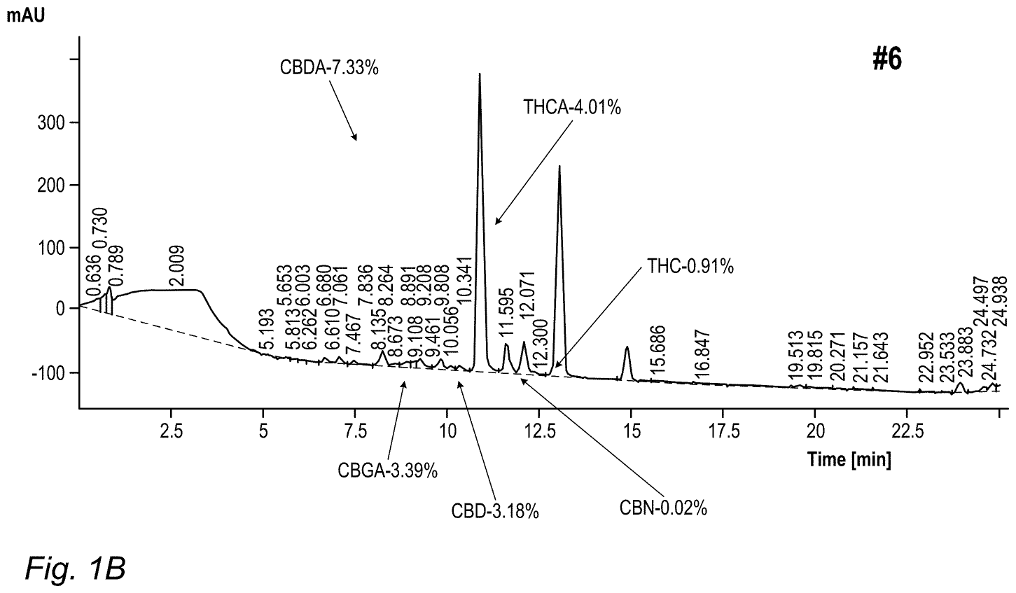

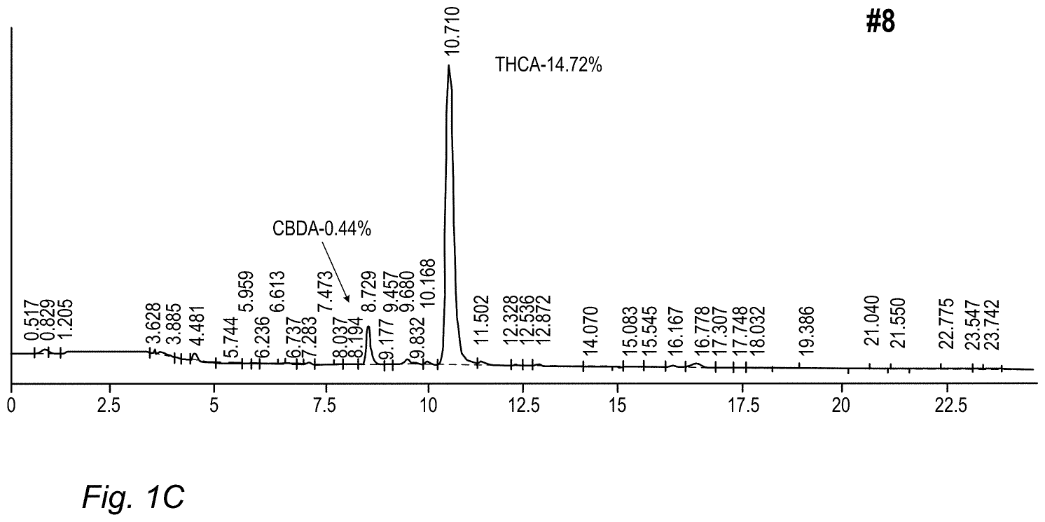

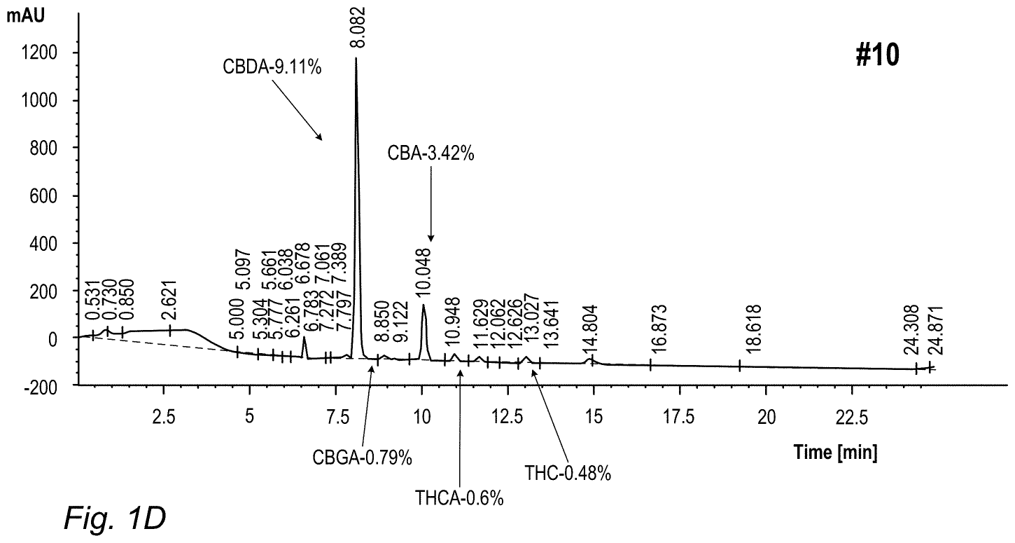

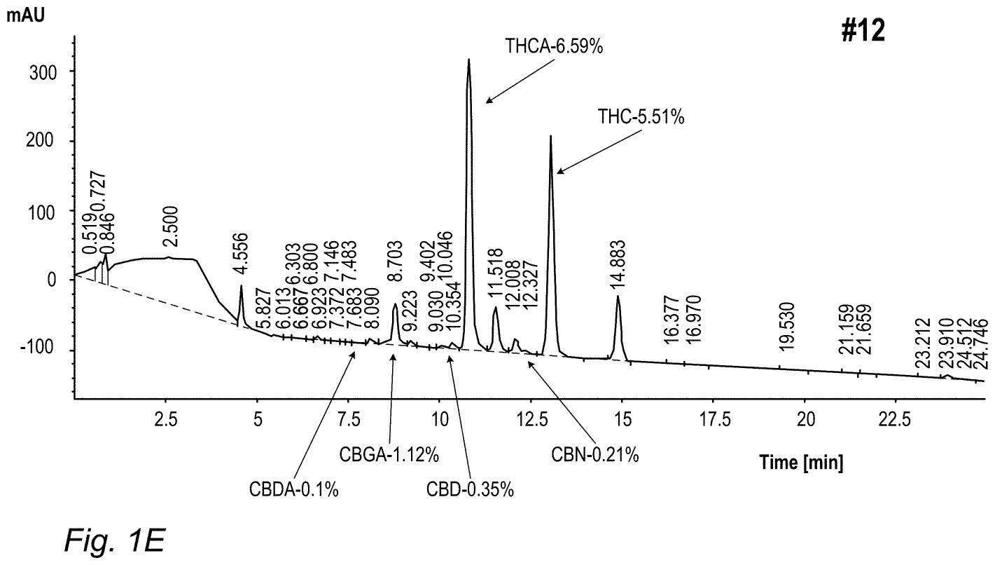

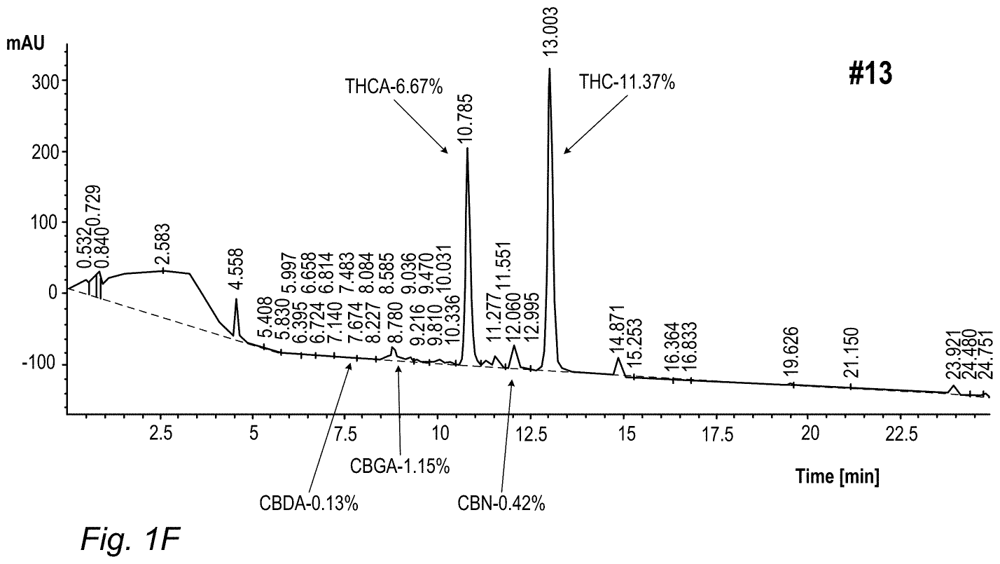

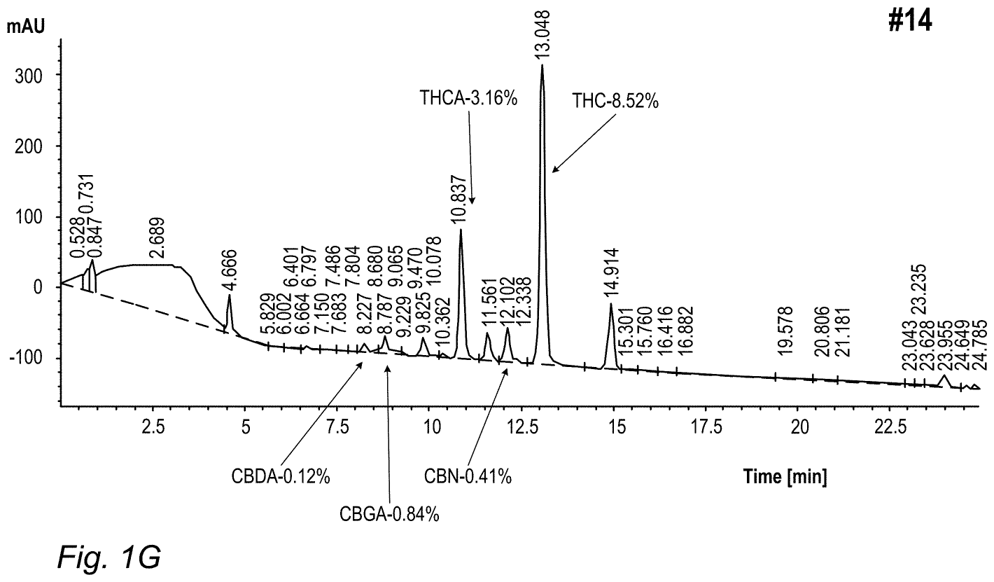

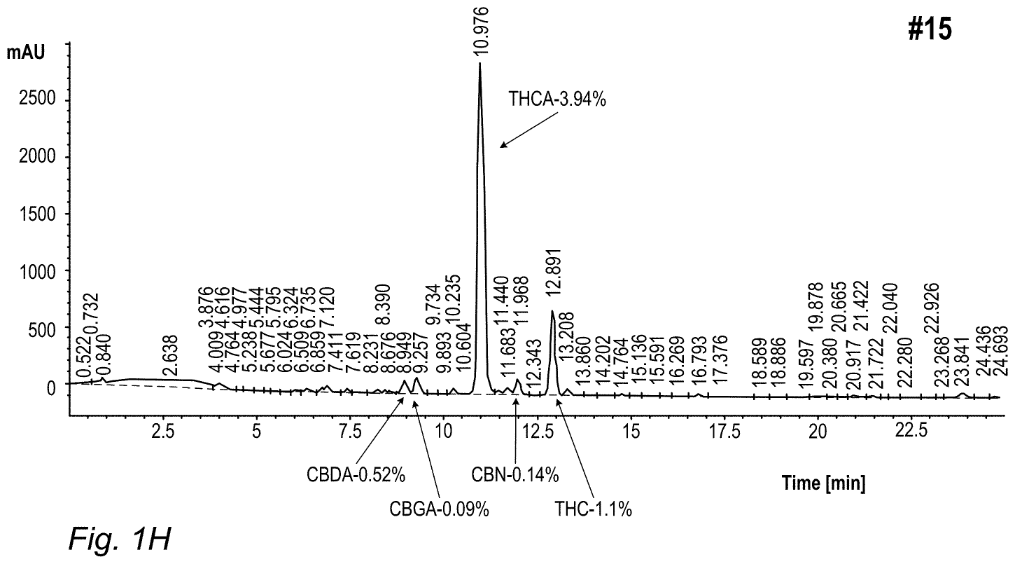

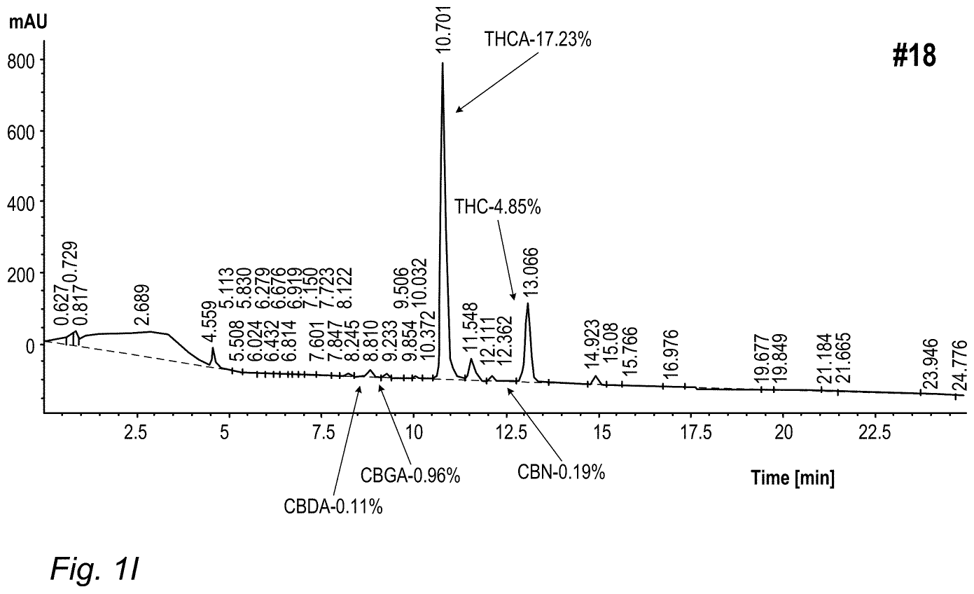

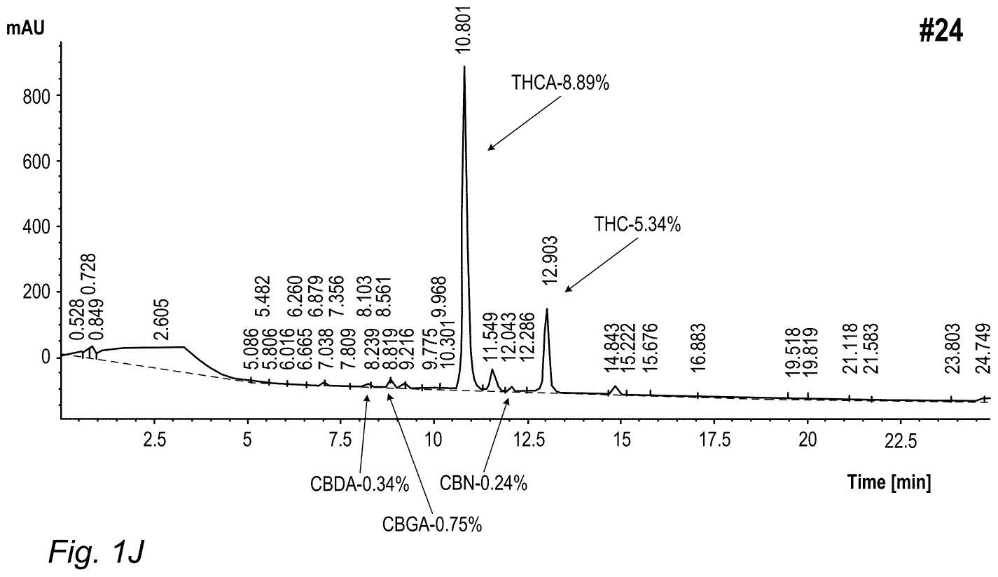

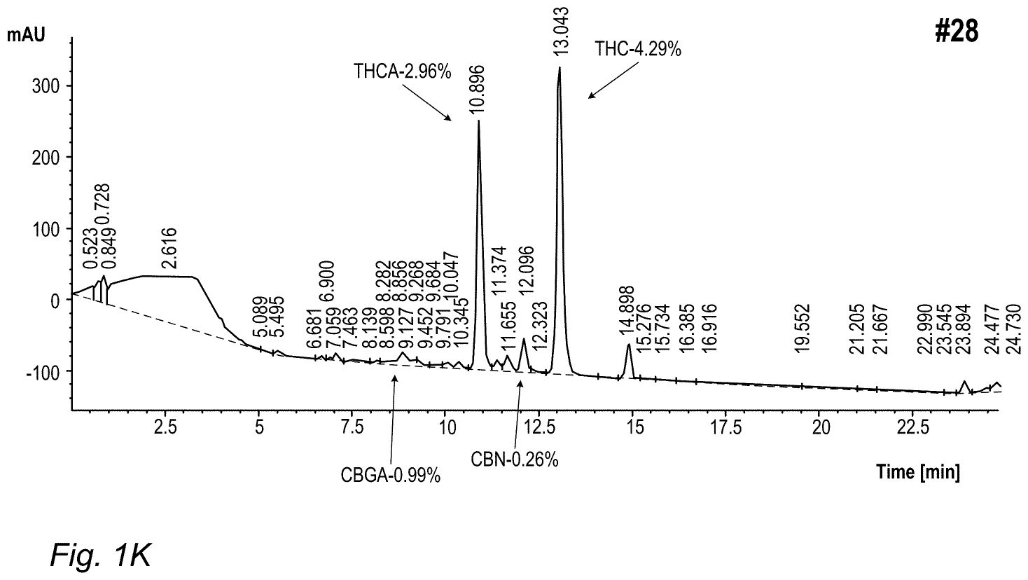

FIGS. 1A-1M. High performance liquid chromatography (HPLC) profiles of tested lines, in accordance with some embodiments of the present invention.

FIG. 1A. Chromatogram of #4 extract. Total THC equivalent 10.44% and CBD 0.38%.

FIG. 1B. Chromatogram of #6 extract. Total THC equivalent 4.43% and CBD 9.61%.

FIG. 1C. Chromatogram of #8 extract. Total THC equivalent 14.72% and CBD 0.41%.

FIG. 1D. Chromatogram of #10 extract. Total THC equivalent 1.0% and CBD 11.41%.

FIG. 1E. Chromatogram of #12 extract. Total THC equivalent 12.29% and CBD 0.44%.

FIG. 1F. Chromatogram of #13 extract. THC equivalent 17.22% and CBD 0.21%.

FIG. 1G. Chromatogram of #14 extract. Total THC equivalent 11.3% and CBD 0.42%.

FIG. 1H. Chromatogram of #15 extract. Total THC equivalent 4.57% and CBD 0.47%.

FIG. 1I. Chromatogram of #18 extract. Total THC equivalent 19.96% and CBD 0.1%.

FIG. 1J. Chromatogram of #24 extract. THC equivalent 13.13% and CBD 0.64%.

FIG. 1K. Chromatogram of #28 extract. Total THC equivalent 6.89% and CBD 0.1%.

FIG. 1L. Chromatogram of #30 extract. Total THC equivalent 9.93% and CBD 0.05%.

FIG. 1M. Chromatogram of #81 extract. Total THC equivalent 1.37% and CBD 10.38%.

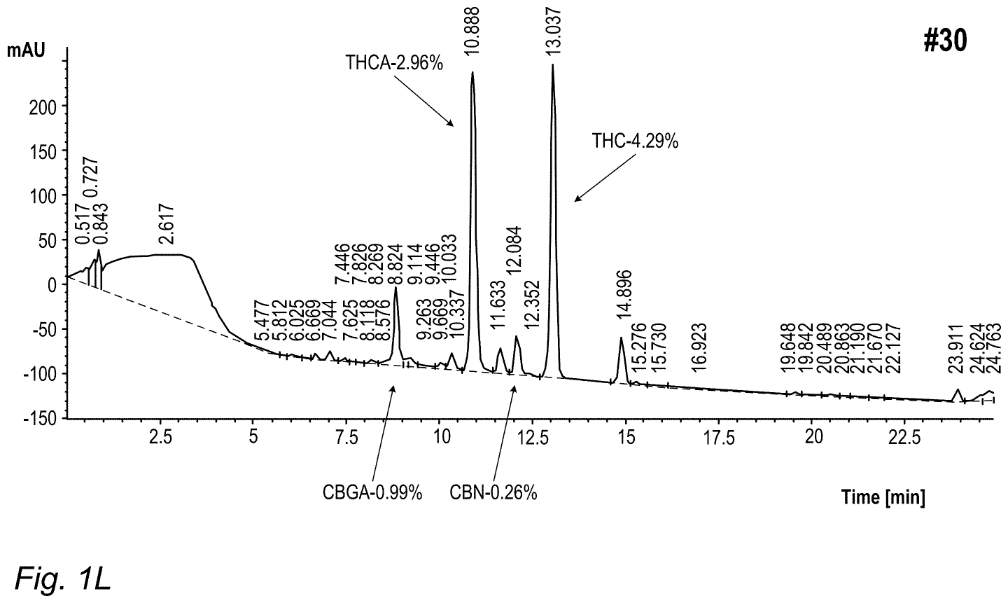

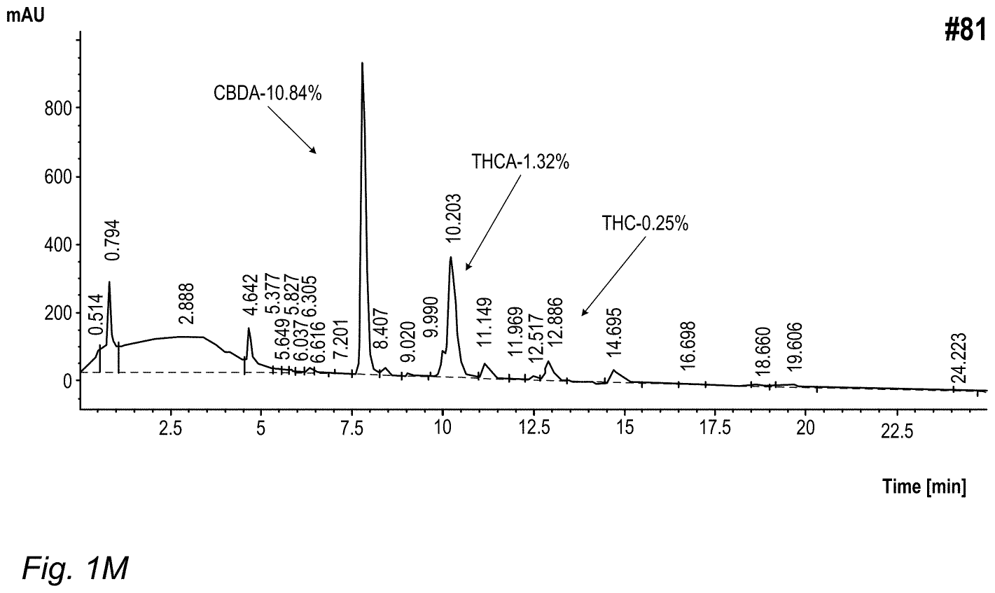

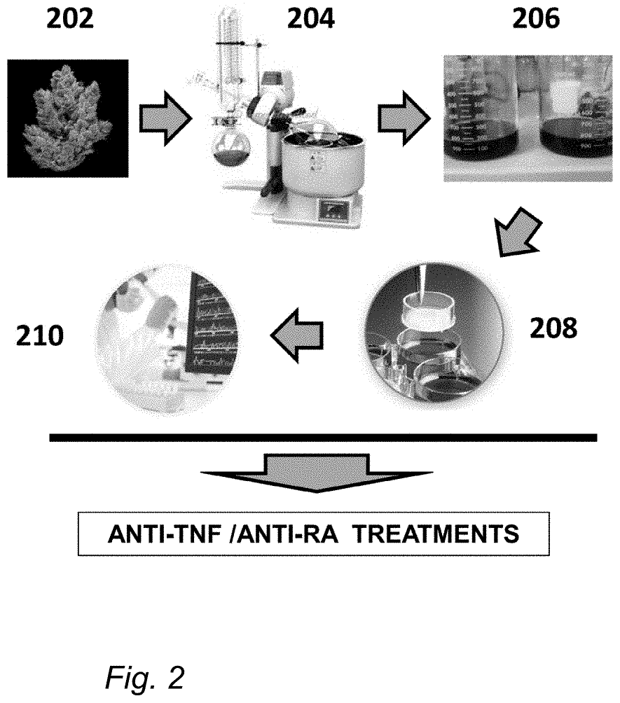

FIG. 2 shows a scheme of the approach and method to identify new anti-inflammatory lines and extracts. New cannabis cultivars were used for extract preparations. The extracts were further tested for their anti-inflammatory activity using human 3D tissues and global transcriptome profiling that revealed new cannabis lines with anti-TNF activity and anti-RA activity.

Step 202—a cultivar growing step. Around 250 unique marijuana and around 120 unique hemp cultivars were used to generate approximately 1,200 marijuana/marijuana, hemp/hemp and hemp/marijuana hybrids. Cultivars are typically grown in soil/vermiculite (2:1) mix. First, plants are grown under 16h day, 8h night for approximately 6 weeks when they were moved to another grow room and grown at 12 h day and 12 h night for another 6-8 weeks until they developed mature flowers. In both rooms, they were grown under the high pressure sodium (HPS) lights of ˜400 W/m2. Collected flowers were then tested for cannabinoids and terpenoids and those with most diversity in composition, or those that had highest amount of one or more cannabinoid or terpenoid or those that had the presence of unique terpenoids were used for breeding. The progeny of these crosses was then grown and further tested for cannabinoids/terpenoids as well as for growth parameters, such as height, response to nutrients, responses to pathogens, amongst others. In some cases, these plants were then crossed again using siblings with similar traits (cannabinoids/terpenoids for example). The seeds of these cultivars (resulting from crosses) are stored at +4° C. in the fridge in the locked cage. Approximately 600 cultivars with the best parameters, such as diversity of cannabinoids and terpenoids, plant growth vigor (germination rate, mutation time, yield of flowers, nutrients response, response to pathogens, size of flowers) and other features such as distinct smell for example were germinated and approximately 400 extracts were made.

Step 204, an extraction step, such as organic solvent extraction. Most solvents can be used. In one experiment ethyl acetate was used. This should not be deemed as limiting. For extract preparation, 3 g of the powdered flower tissue were used in 100 ml of ethyl acetate in a 250 mL Erlenmeyer flask. The flasks were then wrapped with tin foil and shaken continuously (120 rpm) in an incubator at 21° C. overnight and in the dark. After overnight solvent extraction the extracts were filtered through cotton into a 100 ml round bottom flask. The extracts were concentrated to around 2-3 ml using a rotary vacuum evaporator. The extracts were then transferred to a tared 3 dram vial.

Step 206, an evaporation step. The leftover solvent was evaporated to dryness in an oven overnight at 50° C. to eliminate the solvent completely. Mass of each extract was recorded, and the extracts were stored at −20° C. The stocks were prepared weighing a 3-6 mg of crude extract into a micro centrifuge tube. The crude extract was dissolved in DMSO (Dimethyl sulfoxide anhydrous) to reach 60 mg/mL final concentration and stored at −20° C. Around 400 solvent-based crude extracts of flowers were thus generated.

In an extract biological assay step 208, many of the selected extracts were tested as follows. Appropriate cell culture media (for example RPMI+10% FBS or EMEM+10% FBS) was used to dilute the 60 mg/mL stock. Appropriate amounts of stock extract were added to the media used for 3D tissues, mixed thoroughly to ensure they are in solution and filtered through a 0.22 um syringe filter. These filtrates were ready to be applied to 3D tissues and tested. For example, to achieve the concentration of 0.007 mg/ml, 2.45 μl of stock extract (60 mg/ml) was added to 21 ml of medium.

In a gene expression data analysis step 210, gene expression data were obtained from harvested tissue and altered pathways were analyzed bioinformatically.

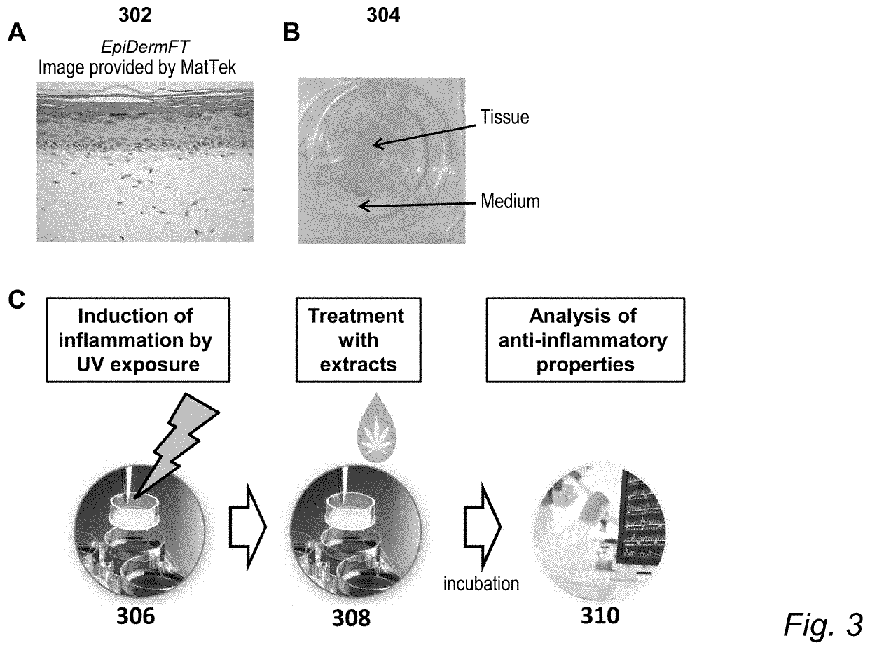

FIG. 3 shows EpiDermFt tissues and experimental set-up. A. EpiDermFT has normal skin tissue structure with differentiated dermis and epidermis and is constructed from human-derived epidermal keratinocytes and dermal fibroblasts. It exhibits in vivo-like growth and morphological characteristics whereby cells sustain differentiation and metabolic status similar to those of human epidermis. B. Tissue insert in a well with medium. C. Scheme of UV-induced inflammation experiment.

In tissue preparation step 302—3D EpiDemFt tissues of normal skin epithelial tissues were used.

Further in step 304,3D tissues are inserted in a well with medium. Tissues were equilibrated in EFT-400 for 24 h (overnight) then culture media EFT-400 was replaced and incubated for another 24 h.

In UV exposure step 306, tissues were exposed for 2 min to UVC, in a biosafety cabinet. Distance from the light source was set to 10 cm.

In extract treatment step 308, all crude extracts were diluted from a 60 mg/mL stock (the stock is prepared in DMSO). For this experiment, a final concentration of 0.01 ug/uL in 30% glycerol-PBS was used. 24 h after UV exposure, 15 uL of 0.01 ug/uL extract solution or control (PBS alone) were applied to the tissue after exposure. Control samples consisted of the following samples: PBS-exposed only, PBS and DMSO added after UVC exposure (crude extracts are stored in DMSO).

In the analysis step 310, the samples were harvested and used for the analysis of mRNA by sequencing. Bioinformatics analysis of mRNA revealed changes in biological pathways associated with inflammation. Extracts with most pronounced changes were identified, including #4, #6, #8, #13 and #14.

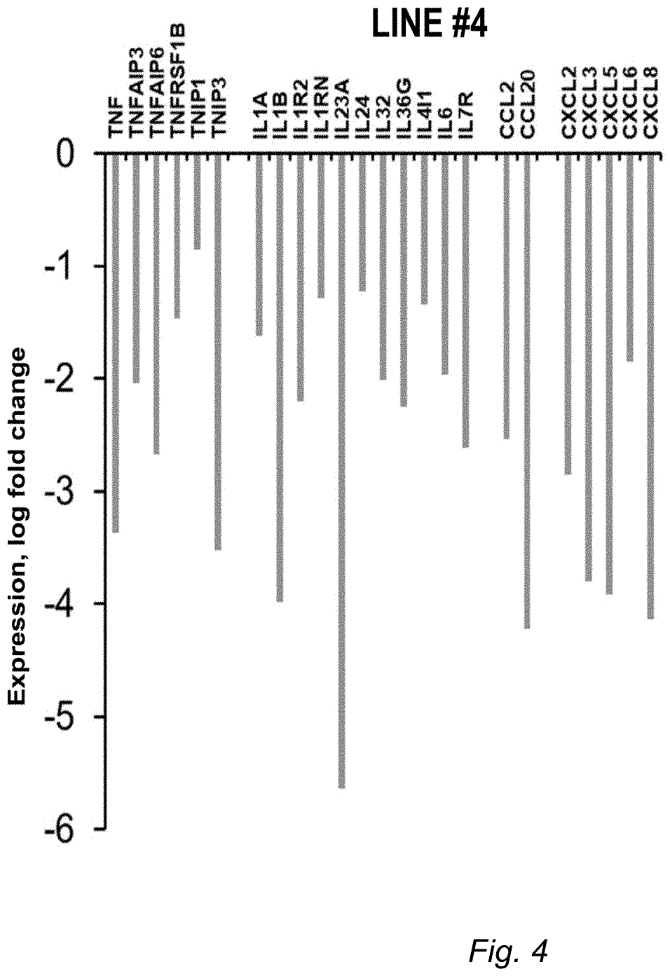

FIG. 4 shows the effects of line #4 expression of TNF, IL and other pro-inflammatory molecules. Genes with a False Discovery Rate (FDR)—adjusted p-value<0.05 and log2 fold change>0.6 (1.5× change) were considered differentially expressed. Changes in the levels of gene expression are shown as log2 fold as compared to control.

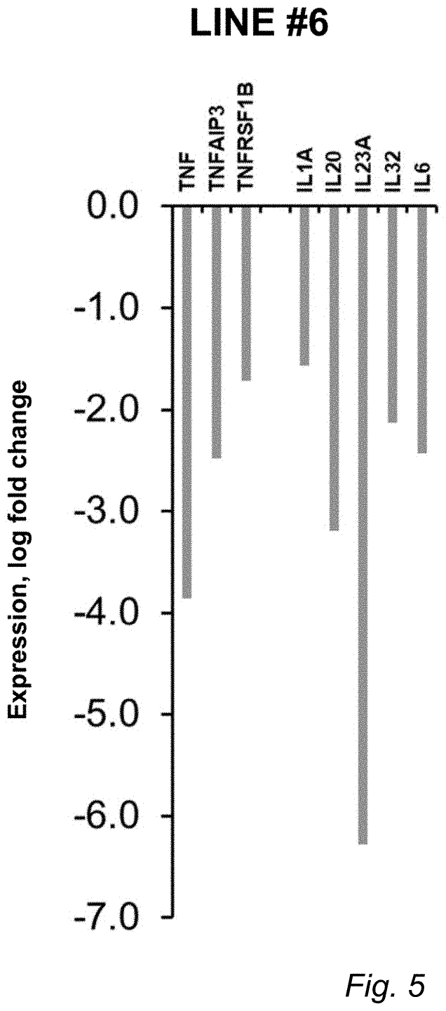

FIG. 5 shows effects of line #6 on the expression of TNF, IL and other pro-inflammatory molecules. Genes with a False Discovery Rate (FDR)-adjusted p-value<0.05 and log2 fold change>0.6 (1.5× change) were considered differentially expressed. Changes in the levels of gene expression are shown as log2 fold as compared to control.

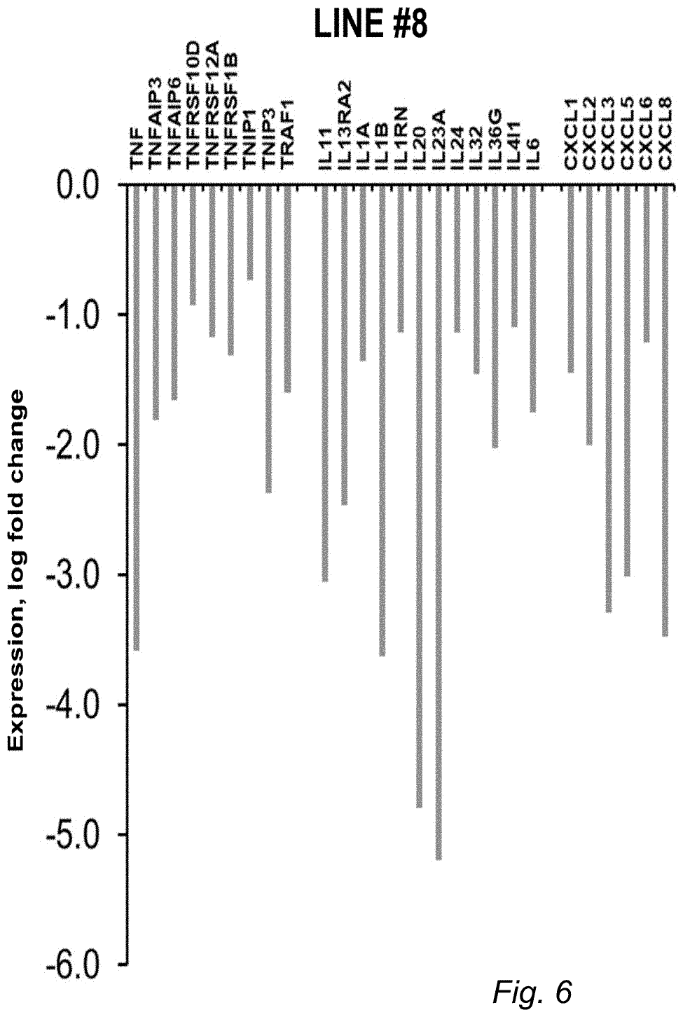

FIG. 6 shows effects of line #8 on the expression of TNF, IL and other pro-inflammatory molecules. Genes with a False Discovery Rate (FDR)-adjusted p-value<0.05 and log2 fold change>0.6 (1.5× change) were considered differentially expressed. Changes in the levels of gene expression are shown as log2 fold as compared to control.

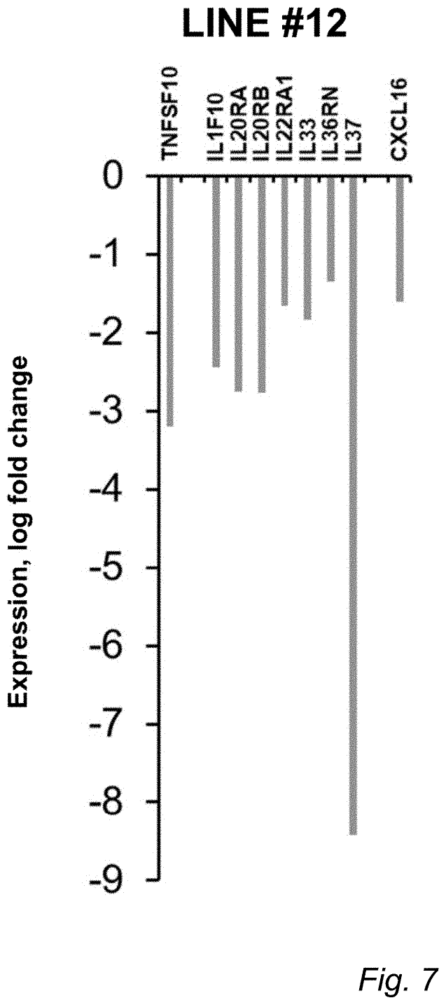

FIG. 7 shows effects of line #12 on the expression of TNF, IL and other pro-inflammatory molecules. Genes with a False Discovery Rate (FDR)-adjusted p-value<0.05 and log2 fold change>0.6 (1.5× change) were considered differentially expressed. Changes in the levels of gene expression are shown as log2 fold as compared to control.

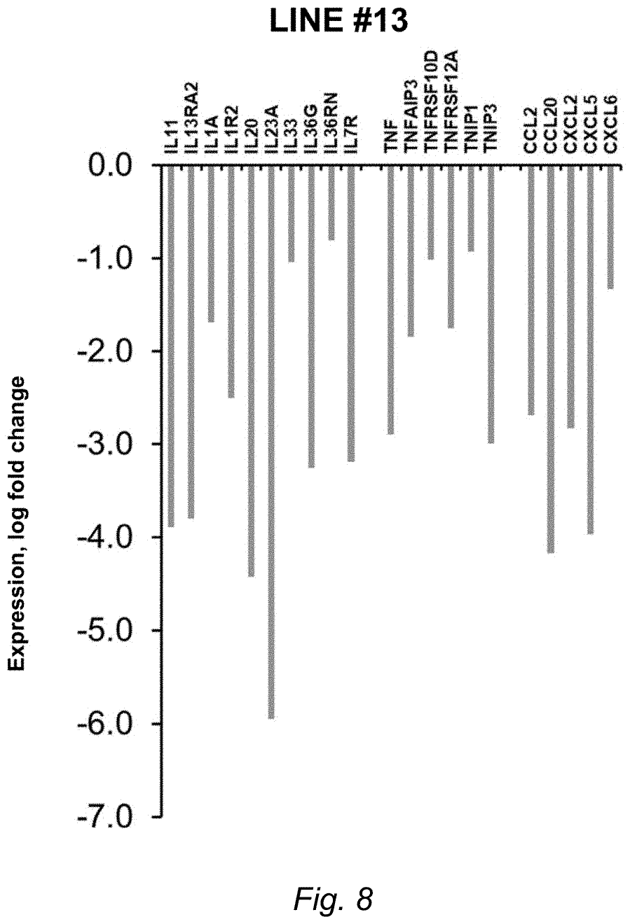

FIG. 8 shows effects of line #13 on the expression of TNF, IL and other pro-inflammatory molecules. Genes with a False Discovery Rate (FDR)-adjusted p-value<0.05 and log2 fold change>0.6 (1.5× change) were considered differentially expressed. Changes in the levels of gene expression are shown as log2 fold as compared to control.

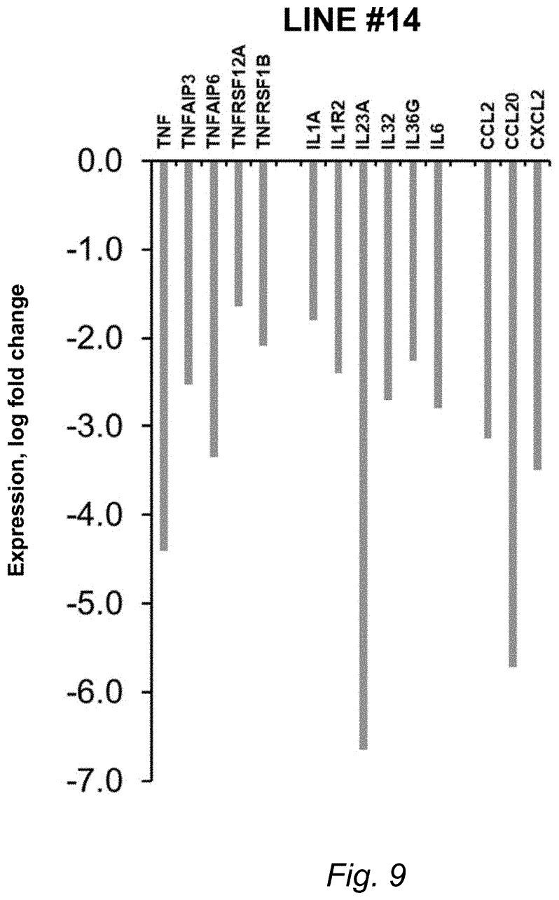

FIG. 9. Shows the effects of line #14 on the expression of TNF, IL and other pro-inflammatory molecules. Genes with a False Discovery Rate (FDR)-adjusted p-value <0.05 and log2 fold change>0.6 (1.5× change) were considered differentially expressed. Changes in the levels of gene expression are shown as log2 fold as compared to control.

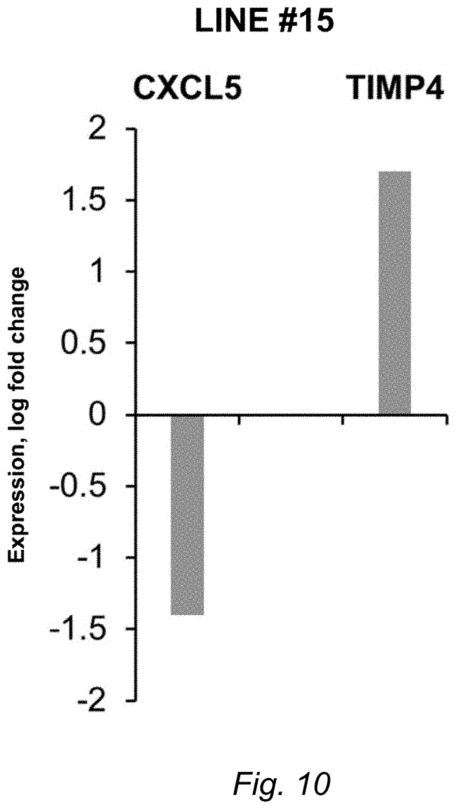

FIG. 10 shows the effects of line #15 on the expression of TNF, IL and other pro-inflammatory molecules. Genes with a False Discovery Rate (FDR)-adjusted p-value<0.05 and log2 fold change>0.6 (1.5× change) were considered differentially expressed. Changes in the levels of gene expression are shown as log2 fold as compared to control.

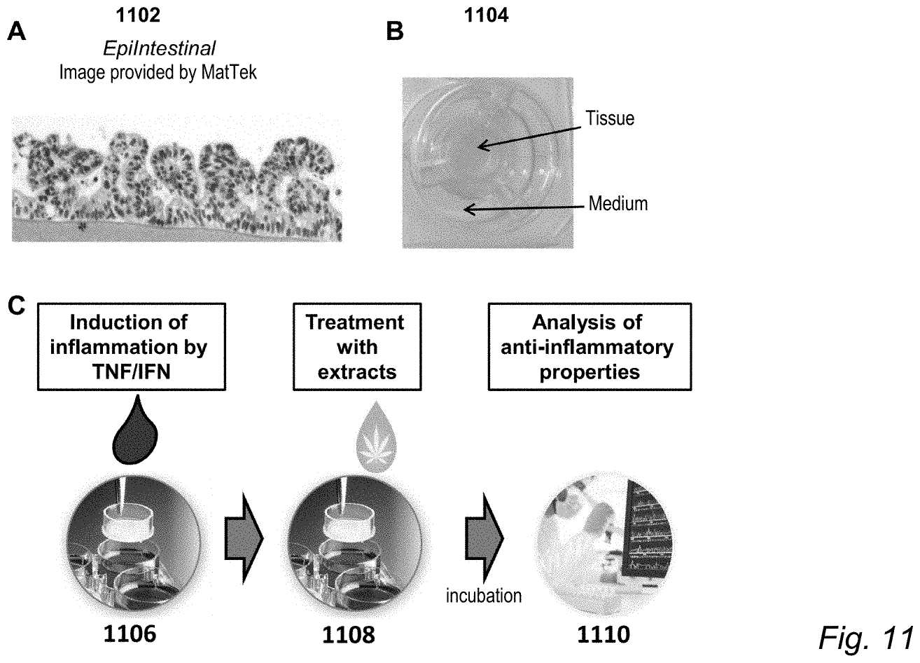

FIG. 11 shows Epilntestinal tissues and experimental method set-up. A. Epilntestinal has normal small intestine epithelial cell structure and is constructed from human-derived columnar epithelial and endothelial cells. It exhibits in vivo-like growth and morphological characteristics whereby cells sustain differentiation and metabolic status similar to those of human intestinal epithelium. B. Tissue insert in a well with medium. C. Scheme of TNF/INF-induced inflammation experiment.

In tissue preparation step 1102—3D Epilntestinal tissues of small intestine epithelial tissues were used.

Further in step 1104, 3D tissues are inserted in a well with medium. Tissues were equilibrated in the medium for 24 h (overnight) then culture medium was replaced and incubated for another 24 h.

In the exposure step 1106, tissues were exposed for 24 h to TNFα (40 ng/ml) and IFNα (5 ng/ml) or to DMSO only.

In extract treatment step 1108, all crude extracts were diluted from a 60 mg/mL stock (the stock is prepared in DMSO). For this experiment, a final concentration of 0.01 μg/μL in 30% glycerol-PBS was used. 15 μL of 0.01 μg/uL extract solution or control (DMSO alone) were applied to the tissue after exposure.

In the analysis step 1110, the samples were harvested and used for the analysis of mRNA by sequencing or of protein by Western blot. Bioinformatics analysis of mRNA revealed changes in biological pathways associated with inflammation. Extracts with most pronounced changes were identified, including #4, #8, #10, #14, #18, #24, #28, #30, #81.

FIGS. 12A-12J show an induction of an inflammation response by TNF/IFN and effects of various extracts on TNF, IL and other pro-inflammatory molecules. Genes with a False Discovery Rate (FDR)-adjusted p-value<0.05 were considered differentially expressed. Changes in the levels of gene expression are shown as log2 fold as compared to control.

FIG. 12A. Induction of expression of TNF, interleukin and chemokine-related genes in response to TNF/IFN.

FIG. 12B. Combined gene expression analysis of genes involved in inflammation (see Table 1 for the full list). “TNF/IFN”—gene expression in response to TNF/IFN (24 h after treatment). “TNF/IFN-DMSO”—persistence of the expression of pro-inflammatory genes 24 h after removal of TNF/IFN and media change (DMSO).

FIGS. 12C-12J. Gene expression in response to various extracts. Extracts were given to cells exposed to TNF/IFN for 24 h. Expression analysis was performed 24 h after exposure to extracts.

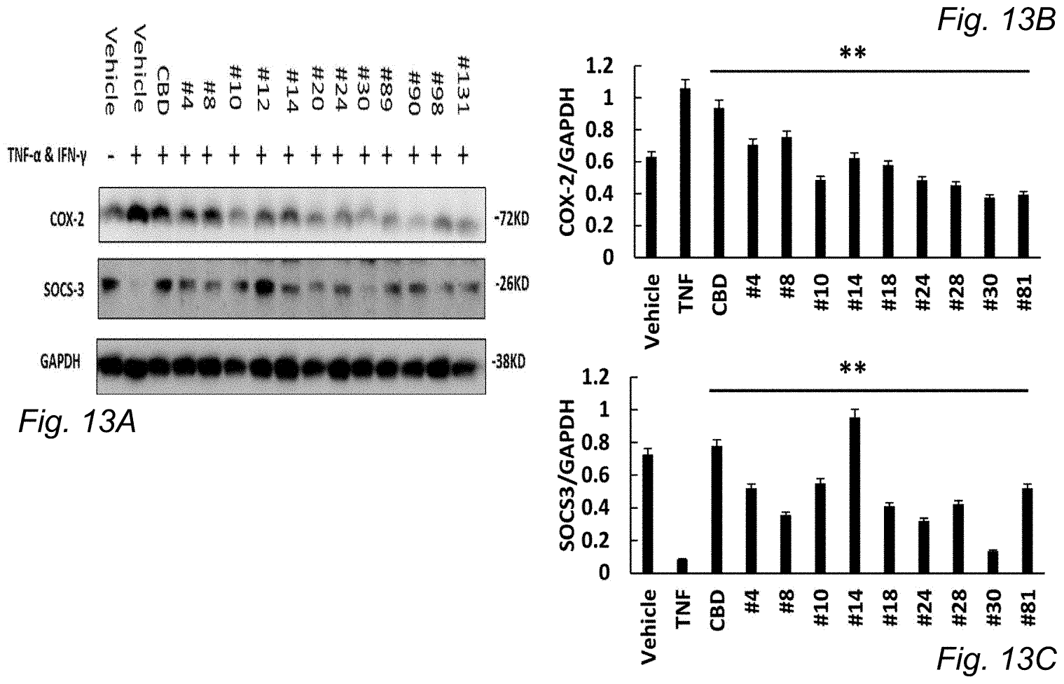

FIG. 13A. is a Western blot analysis of COX-2 and SOCS-3 after the exposure to TNF/IFN and treatment with CBD or crude flower extracts.

HSIEC, human small intestinal epithelial cells were used for the experiments. Cells were grown in normal media (Vehicle-) for 12 h or treated for 6 h with TNFα (40 ng/ml)/IFNγ (5 ng/ml) (Vehicle+) followed by 6h of 10 μ.M CBD or different flower extracts. Cells were then harvested and protein expression was tested.

FIG. 13A is a Western blot image of COX-2, SOCS-3 or GAPDH (used as a control).

FIG. 13B shows a graph of quantification of the data for COX-2 using Image-J. GAPDH was used as a control. Bars shows standard error, calculated from 3 independent blots. Asterisks show significant difference from TNF/IFN induction.

FIG. 13C is a graph showing quantification of the data for SOCS-3 using Image-J. GAPDH was used as a control. Bars shows standard error, calculated from 3 independent blots. Asterisks show significant difference from TNF/IFN induction.

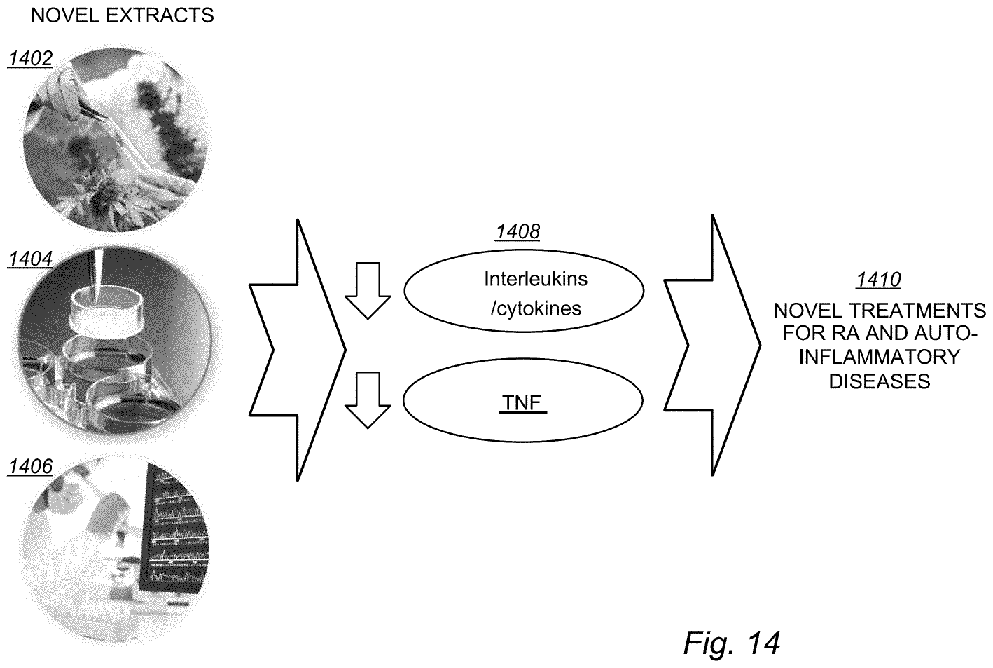

FIG. 14 is a schematic part-pictorial representation of a method for developing novel treatments for RA, autoimmune and inflammatory diseases.

In creating hybrids step 1402, for identification of novel anti-inflammatory extracts hybrids of different cannabis varieties, new hybrids are created and full flower extracts of various hybrids are prepared.

In an inducing inflammation step 1404, skin and intestine epithelial 3D tissues were treated either with UV or with TNF/IFN and subsequently treated with full flower extracts.

In a pro-inflammatory pathway analysis step 1406, the data obtained from mRNA-seq and protein analysis were used to identify downregulated pro-inflammatory pathways.

In an identifying downregulating extracts step 1408, extracts downregulating TNF, interleukins and cytokines are identified.

In a generating new treatments step 1410, such extracts are used for generation of novel treatments for RA and auto-inflammatory diseases.

Described herein are new cannabis lines and their extracts and methods of their use for treating RA and other auto-inflammatory disorders, but is not limited to the steps of: 1) preparation of new cannabis extracts, 2) inducing inflammation in tissues by exposing tissues to UV and 3) modulating the gene expression to cause a reduction of inflammation state in the tissues.

Treatments of UV-exposed tissues with new cannabis extracts significantly affected gene expression leading to profound down-regulation of genes and pathways involved in inflammation, immunity and autoimmunity, especially TNF, IL and cytokines that are main drivers of RA and other auto-inflammatory disorders.

Eleven out of thirteen extracts profoundly down-regulated inflammation genes—TNF, IL, CCL and CXCL genes that are therapeutic targets for the treatment of RA and other auto-inflammatory disorders.

Targeted genes included ILIA, IL1B, IL11,IL6, IL32, IL13RA2, IL1R2, IL20, IL23A, IL33, IL36G, IL36RN, IL7R,IL 37, TNF, TNFAIP3, TNFRSF10D,TNFRSF12A, TNFRSF1B, TNIP1,TNIP3, CCL2,CCL20, CXCL1, CXCL2, CXCL3, CXCL5, CXCL6, CXCL8, CXCL16 (FIGS. 4-10,12,13).

In addition, new extracts may modulate genes and proteins sharing a sequence identity or substantial sequence identity to those genes and proteins listed herein.

Extracts #4, #6, #8, #10, #13, #14, #18, #24, #28, #30 and #81 profoundly down-regulated the molecular pathway that drives RA and other auto-inflammatory diseases. These extracts can be developed into novel therapeutics for RA and other auto-inflammatory diseases (FIG. 14).

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The presented Examples (FIGS. 1-14) are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

TABLE 1
List of genes used for calculation of level
of gene expression in inflammation

| UniProt | Gene symbol | Gene name |
|---|---|---|
| UniProtKB:Q12979 | ABR | Active breakpoint cluster region- |
| | | related protein |
| UniProtKB:P13686 | ACP5 | Tartrate-resistant acid |
| | | phosphatase type 5 |
| UniProtKB:Q04771 | ACVR1 | Activin receptor type-1 |
| UniProtKB:P00813 | ADA | Adenosine deaminase |
| UniProtKB:P30542 | ADORA1 | Adenosine receptor A1 |
| UniProtKB:P29274 | ADORA2A | Adenosine receptor A2a |
| UniProtKB:P29274 | ADORA2A | Adenosine receptor A2a |
| UniProtKB:P29275 | ADORA2B | Adenosine receptor A2b |
| UniProtKB:Q15109 | AGER | Advanced glycosylation end |
| | | product-specific receptor |
| UniProtKB:Q15109 | AGER | Advanced glycosylation end |
| | | product-specific receptor |
| UniProtKB:P50052 | AGTR2 | Type-2 angiotensin II receptor |
| UniProtKB:P50052 | AGTR2 | Type-2 angiotensin II receptor |
| UniProtKB:P23526 | AHCY | Adenosylhomocysteinase |
| UniProtKB:P31749 | AKT1 | RAC-alpha serine/threonine- |
| | | protein kinase |
| UniProtKB:P09917 | ALOX5 | Arachidonate 5-lipoxygenase |
| UniProtKB:P02652 | APOA2 | Apolipoprotein A-II |
| UniProtKB:Q9NR48 | ASH1L | Histone-lysine N- |
| | | methyltransferase ASH1L |
| UniProtKB:P00966 | ASS1 | Argininosuccinate synthase |
| UniProtKB:Q13315 | ATM | Serine-protein kinase ATM |
| UniProtKB:P30530 | AXL | Tyrosine-protein kinase receptor |
| | | UFO |
| UniProtKB:P15291 | B4GALT1 | Beta-1,4-galactosyltransferase 1 |
| UniProtKB:Q9Y5Z0 | BACE2 | Beta-secretase 2 |
| UniProtKB:Q92560 | BAP1 | Ubiquitin carboxyl-terminal |
| | | hydrolase BAP1 |
| UniProtKB:P11274 | BCR | Breakpoint cluster region |
| | | protein |
| UniProtKB:P46663 | BDKRB1 | B1 bradykinin receptor |
| UniProtKB:P22004 | BMP6 | Bone morphogenetic protein 6 |
| UniProtKB:O00238 | BMPR1B | Bone morphogenetic protein |
| | | receptor type-1B |
| UniProtKB:Q06187 | BTK | Tyrosine-protein kinase BTK |
| UniProtKB:Q06187 | BTK | Tyrosine-protein kinase BTK |
| UniProtKB:Q06187 | BTK | Tyrosine-protein kinase BTK |
| UniProtKB:Q5T7M4 | C1QTNF12 | Adipolin |
| UniProtKB:P01024 | C3 | Complement C3 |
| UniProtKB:P01024 | C3 | Complement C3 |
| UniProtKB:Q96GV9 | C5orf30 | UNC119-binding protein C5orf30 |
| UniProtKB:P13671 | C6 | Complement component C6 |
| UniProtKB:P01258 | CALCA | Calcitonin |
| UniProtKB:P01258 | CALCA | Calcitonin |
| UniProtKB:Q16602 | CALCRL | Calcitonin gene-related peptide |
| | | type 1 receptor |
| UniProtKB:P51671 | CCL11 | Eotaxin |
| UniProtKB:O00175 | CCL24 | C-C motif chemokine 24 |
| UniProtKB:P51679 | CCR4 | C-C chemokine receptor type 4 |
| UniProtKB:P10747 | CD28 | T-cell-specific surface |
| | | glycoprotein CD28 |
| UniProtKB:P25942 | CD40 | Tumor necrosis factor receptor |
| | | superfamily member 5 |
| UniProtKB:P40200 | CD96 | T-cell surface protein tactile |
| UniProtKB:Q9BWU1 | CDK19 | Cyclin-dependent kinase 19 |
| UniProtKB:Q9UNI1 | CELA1 | Chymotrypsin-like elastase family |
| | | member 1 |
| UniProtKB:O15516 | CLOCK | Circadian locomoter output cycles |
| | | protein kaput |
| UniProtKB:P21554 | CNR1 | Cannabinoid receptor 1 |
| UniProtKB:P21554 | CNR1 | Cannabinoid receptor 1 |
| UniProtKB:P34972 | CNR2 | Cannabinoid receptor 2 |
| UniProtKB:P25025 | CXCR2 | C-X-C chemokine receptor type 2 |
| UniProtKB:P11511 | CYP19A1 | Aromatase |
| UniProtKB:Q9NR63 | CYP26B1 | Cytochrome P450 26B1 |
| UniProtKB:Q9Y271 | CYSLTR1 | Cysteinyl leukotriene receptor 1 |
| UniProtKB:Q9NRR4 | DROSHA | Ribonuclease 3 |
| UniProtKB:Q1HG43 | DUOXA1 | Dual oxidase maturation factor 1 |
| UniProtKB:Q1HG44 | DUOXA2 | Dual oxidase maturation factor 2 |
| UniProtKB:Q9Y6W6 | DUSP10 | Dual specificity protein |
| | | phosphatase 10 |
| UniProtKB:Q16610 | ECM1 | Extracellular matrix protein 1 |
| UniProtKB:P24530 | EDNRB | Endothelin receptor type B |
| UniProtKB:P00533 | EGFR | Epidermal growth factor receptor |
| UniProtKB:P00533 | EGFR | Epidermal growth factor receptor |
| UniProtKB:Q9BQI3 | EIF2AK1 | Eukaryotic translation initiation |
| | | factor 2-alpha kinase 1 |
| UniProtKB:P08246 | ELANE | Neutrophil elastase |
| UniProtKB:P29317 | EPHA2 | Ephrin type-A receptor 2 |
| UniProtKB:P01588 | EPO | Erythropoietin |
| UniProtKB:P03372 | ESR1 | Estrogen receptor |
| UniProtKB:P14921 | ETS1 | Protein C-ets-1 |
| UniProtKB:P15090 | FABP4 | Fatty acid-binding protein, |
| | | adipocyte |
| UniProtKB:O15360 | FANCA | Fanconi anemia group A protein |
| UniProtKB:Q9BXW9 | FANCD2 | Fanconi anemia group D2 protein |
| UniProtKB:P30273 | FCER1G | High affinity immunoglobulin |
| | | epsilon receptor subunit gamma |
| UniProtKB:P30273 | FCER1G | High affinity immunoglobulin |
| | | epsilon receptor subunit gamma |
| UniProtKB:P30273 | FCER1G | High affinity immunoglobulin |
| | | epsilon receptor subunit gamma |
| UniProtKB:P30273 | FCER1G | High affinity immunoglobulin |
| | | epsilon receptor subunit gamma |
| UniProtKB:Q12946 | FOXF1 | Forkhead box protein F1 |
| UniProtKB:Q9BZS1 | FOXP3 | Forkhead box protein P3 |
| UniProtKB:P22466 | GAL | Galanin peptides |
| UniProtKB:P23771 | GATA3 | Trans-acting T-cell-specific |
| | | transcription factor GATA-3 |
| UniProtKB:Q13304 | GPR17 | Uracil nucleotide/cysteinyl |
| | | leukotriene receptor |
| UniProtKB:P07203 | GPX1 | Glutathione peroxidase 1 |
| UniProtKB:P36969 | GPX4 | Phospholipid hydroperoxide |
| | | glutathione peroxidase |
| UniProtKB:P81172 | HAMP | Hepcidin |
| UniProtKB:Q30201 | HFE | Hereditary hemochromatosis |
| | | protein |
| UniProtKB:P14210 | HGF | Hepatocyte growth factor |
| UniProtKB:Q96A08 | HIST1H2BA | Histone H2B type 1-A |
| UniProtKB:P10809 | HSPD1 | 60 kDa heat shock protein, |
| | | mitochondrial |
| UniProtKB:P05362 | ICAM1 | Intercellular adhesion molecule 1 |
| UniProtKB:P14902 | IDO1 | Indoleamine 2,3-dioxygenase 1 |
| UniProtKB:P14902 | IDO1 | Indoleamine 2,3-dioxygenase 1 |
| UniProtKB:P22692 | IGFBP4 | Insulin-like growth factor- |
| | | binding protein 4 |
| UniProtKB:P22301 | IL10 | Interleukin-10 |
| UniProtKB:P29460 | IL12B | Interleukin-12 subunit beta |
| UniProtKB:P35225 | IL13 | Interleukin-13 |
| UniProtKB:P40933 | IL15 | Interleukin-15 |
| UniProtKB:Q9UHF5 | IL17B | Interleukin-17B |
| UniProtKB:Q96PD4 | IL17F | Interleukin-17F |
| UniProtKB:Q96F46 | IL17RA | Interleukin-17 receptor A |
| UniProtKB:Q9NRM6 | IL17RB | Interleukin-17 receptor B |
| UniProtKB:Q8NAC3 | IL17RC | Interleukin-17 receptor C |
| UniProtKB:P01583 | IL1A | Interleukin-1 alpha |
| UniProtKB:P14778 | IL1R1 | Interleukin-1 receptor type 1 |
| UniProtKB:P27930 | IL1R2 | Interleukin-1 receptor type 2 |
| UniProtKB:Q01638 | IL1RL1 | Interleukin-1 receptor-like 1 |
| UniProtKB:Q9HB29 | IL1RL2 | Interleukin-1 receptor-like 2 |
| UniProtKB:P60568 | IL2 | Interleukin-2 |
| UniProtKB:Q6UXL0 | IL20RB | Interleukin-20 receptor subunit |
| | | beta |
| UniProtKB:Q6UXL0 | IL20RB | Interleukin-20 receptor subunit |
| | | beta |
| UniProtKB:Q969J5 | IL22RA2 | Interleukin-22 receptor subunit |
| | | alpha-2 |
| UniProtKB:Q9H293 | IL25 | Interleukin-25 |
| UniProtKB:P01589 | IL2RA | Interleukin-2 receptor subunit |
| | | alpha |
| UniProtKB:P01589 | IL2RA | Interleukin-2 receptor subunit |
| | | alpha |
| UniProtKB:Q8NI17 | IL31RA | Interleukin-31 receptor subunit |
| | | alpha |
| UniProtKB:O95760 | IL33 | Interleukin-33 |
| UniProtKB:P05113 | IL5 | Interleukin-5 |
| UniProtKB:Q01344 | IL5RA | Interleukin-5 receptor subunit |
| | | alpha |
| UniProtKB:P17301 | ITGA2 | Integrin alpha-2 |
| UniProtKB:P05107 | ITGB2 | Integrin beta-2 |
| UniProtKB:P18564 | ITGB6 | Integrin beta-6 |
| UniProtKB:O60674 | JAK2 | Tyrosine-protein kinase JAK2 |
| UniProtKB:Q9BX67 | JAM3 | Junctional adhesion molecule C |
| UniProtKB:P05412 | JUN | Transcription factor AP-1 |
| UniProtKB:O15054 | KDM6B | Lysine-specific demethylase 6B |
| UniProtKB:P04264 | KRT1 | Keratin, type II cytoskeletal 1 |
| UniProtKB:P18428 | LBP | Lipopolysaccharide-binding |
| | | protein |
| UniProtKB:P01130 | LDLR | Low-density lipoprotein receptor |
| UniProtKB:P38571 | LIPA | Lysosomal acid lipase/cholesteryl |
| | | ester hydrolase |
| UniProtKB:Q5S007 | LRRK2 | Leucine-rich repeat serine/ |
| | | threonine-protein kinase 2 |
| UniProtKB:P01374 | LTA | Lymphotoxin-alpha |
| UniProtKB:P07948 | LYN | Tyrosine-protein kinase Lyn |
| UniProtKB:P07948 | LYN | Tyrosine-protein kinase Lyn |
| UniProtKB:P46734 | MAP2K3 | Dual specificity mitogen-activated |
| | | protein kinase kinase 3 |
| UniProtKB:P04201 | MAS1 | Proto-oncogene Mas |
| UniProtKB:Q8NEM0 | MCPH1 | Microcephalin |
| UniProtKB:P43490 | NAMPT | Nicotinamide |
| | | phosphoribosyltransferase |
| UniProtKB:Q16236 | NFE2L2 | Nuclear factor erythroid 2-related |
| | | factor 2 |
| UniProtKB:P19838 | NFKB1 | Nuclear factor NF-kappa-B p105 |
| | | subunit |
| UniProtKB:Q9BYH8 | NFKBIZ | NF-kappa-B inhibitor zeta |
| UniProtKB:P59044 | NLRP6 | NACHT, LRR and PYD domains- |
| | | containing protein 6 |
| UniProtKB:Q86UT6 | NLRX1 | NLR family member X1 |
| UniProtKB:P46531 | NOTCH1 | Neurogenic locus notch homolog |
| | | protein 1 |
| UniProtKB:O15130 | NPFF | Pro-FMRFamide-related |
| | | neuropeptide FF |
| UniProtKB:P01160 | NPPA | Natriuretic peptides A |
| UniProtKB:Q15761 | NPY5R | Neuropeptide Y receptor type 5 |
| UniProtKB:Q15761 | NPY5R | Neuropeptide Y receptor type 5 |
| UniProtKB:P21589 | NT5E | 5′-nucleotidase |
| UniProtKB:O60356 | NUPR1 | Nuclear protein 1 |
| UniProtKB:O15527 | OGG1 | N-glycosylase/DNA lyase |
| UniProtKB:P35372 | OPRM1 | Mu-type opioid receptor |
| UniProtKB:P51575 | P2RX1 | P2X purinoceptor 1 |
| UniProtKB:Q99572 | P2RX7 | P2X purinoceptor 7 |
| UniProtKB:Q96KB5 | PBK | Lymphokine-activated killer |
| | | T-cell-originated protein kinase |
| UniProtKB:O75594 | PGLYRP1 | Peptidoglycan recognition |
| | | protein 1 |
| UniProtKB:Q96PD5 | PGLYRP2 | N-acetylmuramoyl-L-alanine |
| | | amidase |
| UniProtKB:P48736 | PIK3CG | Phosphatidylinositol 4,5- |
| | | bisphosphate 3-kinase catalytic |
| | | subunit gamma isoform |
| UniProtKB:Q9Y263 | PLAA | Phospholipase A-2-activating |
| | | protein |
| UniProtKB:P60201 | PLP1 | Myelin proteolipid protein |
| UniProtKB:P06746 | POLB | DNA polymerase beta |
| UniProtKB:P37231 | PPARG | Peroxisome proliferator-activated |
| | | receptor gamma |
| UniProtKB:P42785 | PRCP | Lysosomal Pro-X carboxypeptidase |
| UniProtKB:P28070 | PSMB4 | Proteasome subunit beta type-4 |
| UniProtKB:P25105 | PTAFR | Platelet-activating factor |
| | | receptor |
| UniProtKB:O14684 | PTGES | Prostaglandin E synthase |
| UniProtKB:O14684 | PTGES | Prostaglandin E synthase |
| UniProtKB:P35354 | PTGS2 | Prostaglandin G/H synthase 2 |
| UniProtKB:Q9ULZ3 | PYCARD | Apoptosis-associated speck-like |
| | | protein containing a CARD |
| UniProtKB:O95267 | RASGRP1 | RAS guanyl-releasing protein 1 |
| UniProtKB:Q06330 | RBPJ | Recombining binding protein |
| | | suppressor of hairless |
| UniProtKB:Q9Y3P4 | RHBDD3 | Rhomboid domain-containing |
| | | protein 3 |
| UniProtKB:Q6R327 | RICTOR | Rapamycin-insensitive companion |
| | | of mTOR |
| UniProtKB:P05109 | S100A8 | Protein S100-A8 |
| UniProtKB:P05109 | S100A8 | Protein S100-A8 |
| UniProtKB:Q99500 | S1PR3 | Sphingosine 1-phosphate |
| | | receptor 3 |
| UniProtKB:Q15858 | SCN9A | Sodium channel protein type 9 |
| | | subunit alpha |
| UniProtKB:P18827 | SDC1 | Syndecan-1 |
| UniProtKB:Q96EE3 | SEH1L | Nucleoporin SEH1 |
| UniProtKB:P16109 | SELP | P-selectin |
| UniProtKB:P01008 | SERPINC1 | Antithrombin-III |
| UniProtKB:P36955 | SERPINF1 | Pigment epithelium-derived |
| | | factor |
| UniProtKB:Q86VZ5 | SGMS1 | Phosphatidylcholine:ceramide |
| | | cholinephosphotransferase 1 |
| UniProtKB:P52569 | SLC7A2 | Cationic amino acid transporter 2 |
| UniProtKB:P52569 | SLC7A2 | Cationic amino acid transporter 2 |
| UniProtKB:Q15797 | SMAD1 | Mothers against decapentaplegic |
| | | homolog 1 |
| UniProtKB:P84022 | SMAD3 | Mothers against decapentaplegic |
| | | homolog 3 |
| UniProtKB:Q99835 | SMO | Smoothened homolog |
| UniProtKB:O14543 | SOCS3 | Suppressor of cytokine signaling 3 |
| UniProtKB:O75159 | SOCS5 | Suppressor of cytokine signaling 5 |
| UniProtKB:Q9NYA1 | SPHK1 | Sphingosine kinase 1 |
| UniProtKB:P10451 | SPP1 | Osteopontin |
| UniProtKB:P40763 | STAT3 | Signal transducer and activator of |
| | | transcription 3 |
| UniProtKB:P51692 | STAT5B | Signal transducer and activator of |
| | | transcription 5B |
| UniProtKB:Q9UEW8 | STK39 | STE20/SPS1-related proline- |
| | | alanine-rich protein kinase |
| UniProtKB:Q9BXA5 | SUCNR1 | Succinate receptor 1 |
| UniProtKB:P20366 | TAC1 | Protachykinin-1 |
| UniProtKB:Q9NUY8 | TBC1D23 | TBC1 domain family member 23 |
| UniProtKB:Q9UP52 | TFR2 | Transferrin receptor protein 2 |
| UniProtKB:P21980 | TGM2 | Protein-glutamine gamma- |
| | | glutamyltransferase 2 |
| UniProtKB:P01033 | TIMP1 | Metalloproteinase inhibitor 1 |
| UniProtKB:O60603 | TLR2 | Toll-like receptor 2 |
| UniProtKB:O15455 | TLR3 | Toll-like receptor 3 |
| UniProtKB:O00206 | TLR4 | Toll-like receptor 4 |
| UniProtKB:Q9Y2C9 | TLR6 | Toll-like receptor 6 |
| UniProtKB:Q9NYK1 | TLR7 | Toll-like receptor 7 |
| UniProtKB:Q9NR97 | TLR8 | Toll-like receptor 8 |
| UniProtKB:P01375 | TNF | Tumor necrosis factor |
| UniProtKB:P21580 | TNFAIP3 | Tumor necrosis factor alpha- |
| | | induced protein 3 |
| UniProtKB:P20333 | TNFRSF1B | Tumor necrosis factor receptor |
| | | superfamily member 1B |
| UniProtKB:P20333 | TNFRSF1B | Tumor necrosis factor receptor |
| | | superfamily member 1B |
| UniProtKB:Q8NER1 | TRPV1 | Transient receptor potential |
| | | cation channel subfamily V |
| | | member 1 |
| UniProtKB:Q8NER1 | TRPV1 | Transient receptor potential |
| | | cation channel subfamily V |
| | | member 1 |
| UniProtKB:Q9HBA0 | TRPV4 | Transient receptor potential |
| | | cation channel subfamily V |
| | | member 4 |
| UniProtKB:O60636 | TSPAN2 | Tetraspanin-2 |
| UniProtKB:O75896 | TUSC2 | Tumor suppressor candidate 2 |
| UniProtKB:P55089 | UCN | Urocortin |
| UniProtKB:P22309 | UGT1A1 | UDP-glucuronosyltransferase 1-1 |
| UniProtKB:Q70J99 | UNC13D | Protein unc-13 homolog D |
| UniProtKB:P19320 | VCAM1 | Vascular cell adhesion protein 1 |
| UniProtKB:P19320 | VCAM1 | Vascular cell adhesion protein 1 |
| UniProtKB:Q9HC57 | WFDC1 | WAP four-disulfide core domain |
| | | protein 1 |
| UniProtKB:Q15942 | ZYX | Zyxin |

### Dosage Forms

The compositions of the present invention may be provided in any suitable dosage form. According to some embodiments, the dosage form is an oral dosage form. Oral dosage forms comprise liquids (solutions, suspensions, and emulsions), semi-solids (pastes), and solids (tablets, capsules, powders, granules, premixes, and medicated blocks).

Some examples of oral dosage forms in the art include, WO90/04391, which discloses an oral dosage form of omega-3 polyunsaturated acids to overcome the problems of diseases. It is known to supply said acids in soft gelatine capsule shells.

EP 2 240 581 B1 discloses a gelatine capsule for pharmaceutical use with a controlled release of active ingredients and a process for the preparation of said gelatine capsules. During said process xylose is added to the liquid gelatine from which afterwards gelatine capsules are formed. Gelatine capsules manufactured according to the process provide retarded release of active ingredients.

U.S. Pat. No. 7,264,824 discloses and oral dosage form for food and food supplements, as well as dietetics comprising polyunsaturated acids in a xylose-hardened gelatine capsule with a retarded release time.

According to some embodiments of the present invention, the compositions described herein may be in a suspension or emulsion.

A suspension is a coarse dispersion of insoluble drug particles, generally with a diameter exceeding 1 μm, in a liquid (usually aqueous) medium. Suspensions are useful for administering insoluble or poorly soluble drugs/components or in situations when the presence of a finely divided form of the material in the GI tract is required. The taste of most drugs is less noticeable in suspension than in solution, due to the drug being less soluble in suspension. Particle size is an important determinant of the dissolution rate and bioavailability of drugs in suspension. In addition to the excipients described above for solutions, suspensions include surfactants and thickening agents. Surfactants wet the solid particles, thereby ensuring the particles disperse readily throughout the liquid. Thickening agents reduce the rate at which particles settle to the bottom of the container. Some settling is acceptable, provided the sediment can be readily dispersed when the container is shaken. Because hard masses of sediment do not satisfy this criterion, caking of suspensions is not acceptable.

An emulsion is a system consisting of 2 immiscible liquid phases, one of which is dispersed throughout the other in the form of fine droplets; droplet diameter generally ranges from 0.1-100 μm. The 2 phases of an emulsion are known as the dispersed phase and the continuous phase. Emulsions are inherently unstable and are stabilized through the use of an emulsifying agent, which prevents coalescence of the dispersed droplets. Creaming, as occurs with milk, also occurs with pharmaceutical emulsions. However, it is not a serious problem because a uniform dispersion returns upon shaking. Creaming is, nonetheless, undesirable because it is associated with an increased likelihood of the droplets coalescing and the emulsion breaking. Other additives include buffers, antioxidants, and preservatives. Emulsions for oral administration are usually oil (the active ingredient) in water, and facilitate the administration of oily substances such as castor oil or liquid paraffin in a more palatable form.

A paste is a 2-component semi-solid in which drug is dispersed as a powder in an aqueous or fatty base. The particle size of the active ingredient in pastes can be as large as 100 μm. The vehicle containing the drug may be water; a polyhydroxy liquid such as glycerin, propylene glycol, or polyethylene glycol; a vegetable oil; or a mineral oil. Other formulation excipients include thickening agents, cosolvents, adsorbents, humectants, and preservatives. The thickening agent may be a naturally occurring material such as acacia or tragacanth, or a synthetic or chemically modified derivative such as xanthum gum or hydroxypropylmethyl cellulose. The degree of cohesiveness, plasticity, and syringeability of pastes is attributed to the thickening agent. It may be necessary to include a cosolvent to increase the solubility of the drug. Syneresis of pastes is a form of instability in which the solid and liquid components of the formulation separate over time; it is prevented by including an adsorbent such as microcrystalline cellulose. A humectant (eg, glycerin or propylene glycol) is used to prevent the paste that collects at the nozzle of the dispenser from forming a hard crust. Microbial growth in the formulation is inhibited using a preservative. It is critical that pastes have a pleasant taste or are tasteless.

A tablet consists of one or more active ingredients and numerous excipients and may be a conventional tablet that is swallowed whole, a chewable tablet, or a modified-release tablet (more commonly referred to as a modified-release bolus due to its large unit size). Conventional and chewable tablets are used to administer drugs to dogs and cats, whereas modified-release boluses are administered to cattle, sheep, and goats. The physical and chemical stability of tablets is generally better than that of liquid dosage forms. The main disadvantages of tablets are the bioavailability of poorly water-soluble drugs or poorly absorbed drugs, and the local irritation of the GI mucosa that some drugs may cause.

A capsule is an oral dosage form usually made from gelatin and filled with an active ingredient and excipients. Two common capsule types are available: hard gelatin capsules for solid-fill formulations, and soft gelatin capsules for liquid-fill or semi-solid-fill formulations. Soft gelatin capsules are suitable for formulating poorly water-soluble drugs because they afford good drug release and absorption by the GI tract. Gelatin capsules are frequently more expensive than tablets but have some advantages. For example, particle size is rarely altered during capsule manufacture, and capsules mask the taste and odor of the active ingredient and protect photolabile ingredients.

A powder is a formulation in which a drug powder is mixed with other powdered excipients to produce a final product for oral administration. Powders have better chemical stability than liquids and dissolve faster than tablets or capsules because disintegration is not an issue. This translates into faster absorption for those drugs characterized by dissolution rate-limited absorption. Unpleasant tastes can be more pronounced with powders than with other dosage forms and can be a particular concern with in-feed powders, in which it contributes to variable ingestion of the dose. Moreover, sick animals often eat less and are therefore not amenable to treatment with in-feed powder formulations. Drug powders are principally used prophylactically in feed, or formulated as a soluble powder for addition to drinking water or milk replacer. Powders have also been formulated with emulsifying agents to facilitate their administration as liquid drenches.

A granule is a dosage form consisting of powder particles that have been aggregated to form a larger mass, usually 2-4 mm in diameter. Granulation overcomes segregation of the different particle sizes during storage and/or dose administration, the latter being a potential source of inaccurate dosing. Granules and powders generally behave similarly; however, granules must deaggregate prior to dissolution and absorption.

A premix is a solid dosage form in which an active ingredient, such as a coccidiostat, production enhancer, or nutritional supplement, is formulated with excipients. Premix products are mixed homogeneously with feed at rates (when expressed on an active ingredient basis) that range from a few milligrams to ˜200 g/ton of food/beverage The density, particle size, and geometry of the premix particles should match as closely as possible those of the feed in which the premix will be incorporated to facilitate uniform mixing. Issues such as instability, electrostatic charge, and hygroscopicity must also be addressed. The excipients present in premix formulations include carriers, liquid binders, diluents, anti-caking agents, and anti-dust agents. Carriers, such as wheat middlings, soybean mill run, and rice hulls, bind active ingredients to their surfaces and are important in attaining uniform mixing of the active ingredient. A liquid binding agent, such as a vegetable oil, should be included in the formulation whenever a carrier is used. Diluents increase the bulk of premix formulations, but unlike carriers, do not bind the active ingredients. Examples of diluents include ground limestone, dicalcium phosphate, dextrose, and kaolin. Caking in a premix formulation may be caused by hygroscopic ingredients and is addressed by adding small amounts of anti-caking agents such as calcium silicate, silicon dioxide, and hydrophobic starch. The dust associated with powdered premix formulations can have serious implications for both operator safety and economic losses, and is reduced by including a vegetable oil or light mineral oil in the formulation. An alternate approach to overcoming dust is to granulate the premix formulation.

A medicated block is a compressed feed material that contains an active ingredient, such as a drug, anthelmintic, surfactant (for bloat prevention), or a nutritional supplement, and is commonly packaged in a cardboard box. Ruminants typically have free access to the medicated block over several days, and variable consumption may be problematic. This concern is addressed by ensuring the active ingredient is nontoxic, stable, palatable, and preferably of low solubility. In addition, excipients in the formulation modulate consumption by altering the palatability and/or the hardness of the medicated block. For example, molasses increases palatability and sodium chloride decreases it. Additionally, the incorporation of a binder such as lignin sulfonate in blocks manufactured by compression or magnesium oxide in blocks manufactured by chemical reaction, increases hardness. The hygroscopic nature of molasses in a formulation may also impact the hardness of medicated blocks and is addressed by using appropriate packaging.

In another embodiment, the composition of the present invention is in a chewable oral dosage form. In another embodiment, the chewable oral dosage form is a chewable tablet. In another embodiment, the chewable tablet of the invention is taken slowly by chewing or sucking in the mouth. In another embodiment, the chewable tablet of the invention enables the dried cannabis extracts contained therein to be orally administered without drinking.

According to some embodiments of the present invention, the composition may comprise any suitable flavor or combination of flavors. The composition may further comprise other additives, coloring, emulsifiers. The flavors and additives may be of a natural, semi-synthetic, synthetic source or combinations thereof.

In another embodiment of the present invention, the composition further comprises fructose, sorbitol, microcrystalline cellulose, magnesium stearate, or any combination thereof. In another embodiment, the composition further comprises chamomile. In another embodiment, the composition further comprises ginger. In another embodiment, the composition further comprises peppermint. In another embodiment, the composition further comprises anise. In another embodiment, the composition further comprises fennel. In another embodiment, the composition further comprises thyme. In another embodiment, the composition further comprises Arsenicum album. In another embodiment, the composition further comprises Carbo vegetabilis. In another embodiment, the composition further comprises Ignatia, homeopathic ipecac. In another embodiment, the composition further comprises Nux vomica. In another embodiment, the composition further comprises Zingiber officinale.

In another embodiment, the present invention provides a soft, chewable dosage form which is pliable and chewy, yet dissolves quickly in the mouth, has a long shelf life, contains little moisture which improves stability and decreases the tendency for the dosage form to dry out, does not require cooking or heating as part of the manufacturing process. In another embodiment, the dosage form is used as a matrix for dried cannabis extracts.

In another embodiment, the chewable tablet of the invention comprises a metal salt such as calcium, magnesium, aluminum salt, or any mixture thereof. In another embodiment, the chewable tablet of the invention comprises hydroxyalkyl cellulose. In another embodiment, the chewable tablet of the invention comprises low viscosity hydroxyalkyl cellulose. In another embodiment, the chewable tablet of the invention comprises high viscosity hydroxyalkyl cellulose.

In another embodiment, the chewable tablet of the invention comprises various additives. In another embodiment, the chewable tablet of the invention comprises sweeteners. In another embodiment, the chewable tablet of the invention comprises acidic ingredients. In another embodiment, the chewable tablet of the invention comprises taste correctives. In another embodiment, the chewable tablet of the invention comprises polymeric compounds. In another embodiment, the chewable tablet of the invention comprises essential oils.

In another embodiment, the chewable tablet of the invention is a soft tablet. In another embodiment, the chewable tablet of the invention is made in a state of soft candy. In another embodiment, the chewable tablet of the invention is made in a state of jelly.

In another embodiment, the chewable tablet of the invention comprises a core comprising the vitamins of the invention. In another embodiment, the chewable tablet of the invention comprises an outer layer wrapping the core which is made up of chewable base such as a gum, a soft candy or a caramel.

In another embodiment, the compositions of the present invention may be provided in any suitable food of a solid, semi-solid or liquid form.

The preparation of pharmaceutical compositions that contain a dried cannabis extract, for example by mixing, granulating, or tablet-forming processes, is well understood in the art. The dried cannabis extracts are often mixed with excipients that are pharmaceutically acceptable and compatible with the active ingredient. For oral administration, the active ingredients of compositions of the present invention are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions.

In another embodiment, additional methods of administering the dried cannabis extracts, or compound(s) isolated therefrom, of the invention comprise injectable dosage forms. In another embodiment, the injectable is administered intraperitonealy. In another embodiment, the injectable is administered intramuscularly. In another embodiment, the injectable is administered intradermally. In another embodiment, the injectable is administered intravenously. Each possibility represents a separate embodiment of the present invention.

In another embodiment, the pharmaceutical compositions are administered by intravenous, intra-arterial, or intra-muscular injection of a liquid preparation. Suitable liquid formulations include solutions, suspensions, dispersions, emulsions, oils and the like. In another embodiment, the pharmaceutical compositions are administered intravenously and are thus formulated in a form suitable for intravenous administration. In another embodiment, the pharmaceutical compositions are administered intra-arterially and are thus formulated in a form suitable for intra-arterial administration. In another embodiment, the pharmaceutical compositions are administered intra-muscularly and are thus formulated in a form suitable for intra-muscular administration.

In another embodiment, additional methods of administering the dried cannabis extracts of the invention comprise dispersions, suspensions or emulsions. In another embodiment, the dispersion, suspension or emulsion is administered orally. In another embodiment, the solution is administered by infusion. In another embodiment, the solution is a solution for inhalation. Each possibility represents a separate embodiment of the present invention.

In another embodiment, the pharmaceutical composition is administered as a suppository, for example a rectal suppository or a urethral suppository. In another embodiment, the pharmaceutical composition is administered by subcutaneous implantation of a pellet. In another embodiment, the pellet provides for controlled release of active compound agent over a period of time. Each possibility represents a separate embodiment of the present invention.

In other embodiments, pharmaceutically acceptable carriers for liquid formulations are aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of animal, vegetable, or synthetic origin, for example, peanut oil, soybean oil, olive oil, sunflower oil, fish-liver oil, another marine oil, or a lipid from milk or eggs. Each possibility represents a separate embodiment of the present invention.

In another embodiment, parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions. Examples of oils are those of animal, vegetable, or synthetic origin, for example, peanut oil, soybean oil, olive oil, sunflower oil, fish-liver oil, another marine oil, or a lipid from milk or eggs. Each possibility represents a separate embodiment of the present invention.

In another embodiment, the pharmaceutical compositions provided herein are controlled-release compositions, i.e. compositions in which the active compounds are released over a period of time after administration. Controlled- or sustained-release compositions include formulation in lipophilic depots (e.g. fatty acids, waxes, oils). In another embodiment, the composition is an immediate-release composition, i.e. a composition in which all the active compound is released immediately after administration. Each possibility represents a separate embodiment of the present invention.

In another embodiment, the pharmaceutical composition is delivered in a controlled release system. In another embodiment, the agents are administered using intravenous infusion, an implantable osmotic pump, a transdermal patch, liposomes, or other modes of administration. In another embodiment, a pump is used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials are used; e.g. in microspheres in or an implant. In yet another embodiment, a controlled release system is placed in proximity to the therapeutic target, thus requiring only a fraction of the systemic dose (see, e.g., Goodson, in Medical Applications of Controlled Release, supra, vol. 2, pp. 115-138 (1984); and Langer R, Science 249: 1527-1533 (1990). Each possibility represents a separate embodiment of the present invention.

The compositions of the present invention may comprise one or more additional components may further include an additional component selected from the group consisting of an anti-static agent, a buffering agent, a bulking agent, a chelating agent, a colorant, a diluent, a dye, an emollient, a fragrance, an occlusive agent, a pH-adjusting agent, a preservative, and a vitamin.

The compositions of the present invention may comprise one or more additional active agents, selected from the group consisting of active herbal extracts, analgesics, anti-allergic agents, anti-aging agents, anti-bacterials, antibiotic agents, anticancer agents, antidandruff agents, antidepressants, anti-dermatitis agents, anti-edemics, antihistamines, anti-helminths, anti-hyperkeratolyte agents, anti-inflammatory agents, anti-irritants, anti-microbials, anti-mycotics, anti-proliferative agents, antioxidants, anti-wrinkle agents, anti-pruritics, antiseptic agents, antiviral agents, anti-yeast agents, astringents, topical cardiovascular agents, chemotherapeutic agents, corticosteroids, dicarboxylic acids, disinfectants, fungicides, hair growth regulators, hormones, hydroxy acids, immunosuppressants, immunoregulating agents, keratolytic agents, lactams, metals, metal oxides, mitocides, neuropeptides, non-steroidal anti-inflammatory agents, oxidizing agents, photodynamic therapy agents, retinoids, sanatives, scabicides, self-tanning agents, skin whitening agents, vasoconstrictors, vasodilators, vitamins, vitamin D derivatives and wound healing agents.

According to some embodiments, the composition may comprise one or more anti-oxidants/radical scavengers. The anti-oxidant/radical scavenger may be selected from butylated hydroxy benzoic acids and their salts, coenzyme Q10, coenzyme A, gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts.

In one embodiment, the term “treating” refers to curing a disease. In another embodiment, “treating” refers to preventing a disease. In another embodiment, “treating” refers to reducing the incidence of a disease. In another embodiment, “treating” refers to ameliorating symptoms of a disease. In another embodiment, “treating” refers to inducing remission. In another embodiment, “treating” refers to slowing the progression of a disease.

The references cited herein teach many principles that are applicable to the present invention. Therefore, the full contents of these publications are incorporated by reference herein where appropriate for teachings of additional or alternative details, features and/or technical background.

It is to be understood that the invention is not limited in its application to the details set forth in the description contained herein or illustrated in the drawings. The invention is capable of other embodiments and of being practiced and carried out in various ways. Those skilled in the art will readily appreciate that various modifications and changes can be applied to the embodiments of the invention as hereinbefore described without departing from its scope, defined in and by the appended claims.

### REFERENCES

1. Scott D L, Wolfe F, and Huizinga T W. Rheumatoid arthritis. Lancet, 2010; 376: 1094-108

2. Radner H, Aletaha D. Anti-TNF in rheumatoid arthritis: an overview. Wien Med Wochenschr. 2015; 165(1-2):3-9

3. Hausmann J S. Targeting cytokines to treat autoinflammatory diseases. Clin Immunol. 2018 Oct. 27. pii: S1521-6616(18)30432-7.

4. Wallace K L, Zheng L B, Kanazawa Y, Shih D Q. Immunopathology of inflammatory bowel disease. World J Gastroenterol. 2014 Jan. 7;20(1):6-21.

1. A method for treating inflammation, the method comprising:
a) combining at least one marijuana or hemp cultivar and at least one other marijuana or hemp cultivar to form at least one Cannabis line;
b) extracting at least one compound from said at least one Cannabis line to form an extract; and
c) treating at least one of a mammalian subject and an in vitro model with at least one of said extract and said at least one compound in an effective amount to treat said inflammation.

2. A method according to claim 1, wherein said treating step comprises providing an effective amount of said extract or said at least one compound to said mammalian subject or to said in vitro model to modulate gene expression.

3. A method according to claim 1, wherein said modulation of gene expression comprises modulating at least one gene selected from the group consisting of: a TNF pathway gene, a TNI-R gene, an Interleukin gene, a chemokine gene, a gene associated with an inflammatory disease, a gene associated with an inflammatory disorder, a gene associated with a leukocyte, a gene associated with a body joint, a gene associated with synovial fluid, a gene associated with intestine, and combinations thereof.

4. A method according to claim 3, wherein said modulation of gene expression results in at least one of a reduction or an increase of 0.1-3 log₂ fold change in expression of said at least one gene.

5. A method according to claim 4, wherein said at least one Cannabis line is selected from the group consisting of a marijuana/marijuana hybrid line, hemp/hemp hybrid line and hemp/marijuana hybrid line.

6. A method according to claim 5, wherein said at least one line is selected from the group consisting of designated lines #4, #6, #8, #10, #12, #13, #14, #18, #24, #28, #30 and #81.

7. A method according to claim 1, wherein said extracting step comprises extracting flowers of said at least one Cannabis line.

8. A method according to claim 7, wherein said extracting step comprises extracting said at least one compound in at least one organic solvent.

9. A method according to claim 6, wherein said extracting step is performed at a temperature in the range of 15-to 60° C. and at a pressure in a range of −0.5 to 1.5 bar and wherein said at least one organic solvent comprises ethyl acetate.

10. A method according to claim 4, wherein said at least one gene is selected from the group consisting of: TNF, TNFAIP3, TNFAIP6, TNFRSF1B, TNIP1, TNIP3, ILIA, IL1B, IL1R2, IL1RN, IL23A, IL24, IL32, IL36G, IL411, IL6, IL7R, CCL2, CCL20, CXCL2, CXCL3, CXCLS, CXCL6, CXCL8, COX-2, SOCS-3, STAT-3 and BCL-2 and combinations thereof.

11. A method according to claim 10, wherein said at least one Cannabis line comprises line #4.

12. A method according to claim 4, wherein said at least one gene is selected from the group consisting of: TNF, TNFAIP3, TNFRSF1B, ILIA, IL1B, IL1R2, IL20, IL23A, IL32 and IL6 and combinations thereof.

13. A method according to claim 12, wherein said at least one Cannabis line comprises line #6.

14. A method according to claim 4, wherein said at least one gene is selected from the group consisting of: TNF, TNFAIP3, TNFAIP6, TNFRSF10D, TNFRSF12A, TNFRSF1B, TNIP1, TRAF1, IL11, IL13RA2, ILIA, IL1B, IL1RN, IL20, IL23A, IL24, IL32, IL36G, IL411, IL6, CXCL1, CXCL2, CXCL3, CXCLS, CXCL6, CXCL8, COX-2, SOCS-3, STAT-3, BCL-2 and combinations thereof.

15. A method according to claim 14, wherein said at least one Cannabis line comprises line #8.

16. A method according to claim 4, wherein said at least one gene is selected from the group consisting of: TNI-RSF10, IL1F0, IL20RA, IL20RB, IL22RA1, IL33, IL36RN, IL37 and CXCL16 and combinations thereof.

17. An organic extract of at least one plant line, said at least one plant line formed from combining at least one of:
a) at least one marijuana cultivar or hemp; and
b) at least one other marijuana or hemp cultivar,
wherein said organic extract comprises at least one compound suitable for treating an inflammatory mammalian disease or disorder.

18. An organic extract according to claim 17, wherein said at least one plant line comprises a Cannabis sativa line.

19. An organic extract according to claim 18, wherein said mammalian inflammatory disease or disorder is selected from the group consisting of arthritis, rheumatoid arthritis, psoriatic arthritis, osteoarthritis, Familial Mediterranean Fever, cryopyrin-associated periodic syndrome, is mevalonate kinase, deficiency/hyperimmunoglobulinemia D Syndrome, a TNF receptor-associated autoinflammatory syndrome, systemic juvenile idiopathic arthritis/adult-onset Still's disease, fibromyalgia, Crohn' s disease, ulcerative colitis, inflammation, an allergy and combinations thereof.

20. An organic extract according to claim 19, wherein said organic extract is at least 2-20, 3-15, 4-12, 5-10 or 6-9 times as effective as at least one of THC and CBD, administered at the same concentration in treating said disease.
